(19) United States
(12) Patent Application Publication
(10) Pub. No.: US 2007/0264284 A1
(43) Pub. Date: Nov. 15, 2007

(54) Therapeutic, Prophylactic and Diagnostic Agents for Hepatitis B

(76) Inventors: Stephen Locarnini, Victoria (AU); Kumar Visvanathan, Fitzroy (AU)

Correspondence Address: KNOBBE MARTENS OLSON & BEAR LLP, 2040 MAIN STREET, FOURTEENTH FLOOR, IRVINE, CA, 92614, US

(21) Appl. No.: 11/597,063

(22) PCT Filed: May 19, 2005
(86) PCT No.: PCT/AU05/00716

§ 371 Date: Feb. 27, 2007

(30) Foreign Application Priority Data
May 19, 2004 (AU) 2004902676

(51) Int. Cl. A61K 39/29; C12Q 1/70

(52) U.S. Cl. 424/227.1; 435/5

(57) ABSTRACT

The present invention provides regulation of expression of toll-like receptors by the hepatitis B (HBV) pre-core protein, or its extracellular expression product the hepatitis B E antigen (HbeAg). Compounds regulating such expression have use in the treatment and prophylaxis of HBV infection in animal. The invention also provides methods for diagnosing HBV and agents useful in diagnostic protocols. The present invention further contemplates methods for monitoring disease states in humans and other animal species, including animal models, and providing an indication of the subject for infection by HBV, or development of other diseased states.

### BACKGROUND OF THE INVENTION

1. Field of the Invention

The present invention provides compounds useful in the treatment and prophylaxis of infection in animal species by Hepatitis B virus (HBV). The present invention further provides methods for diagnosing infection by HBV or other disease conditions and agents useful in diagnostic protocols. The present invention further contemplates methods for monitoring disease states in humans and other animal species including animal models and providing an indication of the susceptibility of a subject for infection by HBV or development of other diseased states.

2. Description of the Prior Art

Bibliographic details of the publications referred to in this specification are also collected at the end of the description.

Reference to any prior art in this specification is not, and should not be taken as, an acknowledgment or any form of suggestion that this prior art forms part of the common general knowledge in any country.

Hepatitis B virus (HBV) causes debilitating disease conditions and can lead to acute liver failure. HBV is a DNA virus which replicates via an RNA intermediate and utilizes reverse transcription in its replication strategy. The HBV genome is of a complex nature having a partially double-stranded DNA structure with overlapping open reading frames encoding surface, pre-core, core, polymerase and X genes.

The HBV pre-core/core genes contain two in-frame start codons that control the synthesis of HBcAg (encoded by core gene) and HBeAg (encoded by pre-core gene) that have a co-terminal N-terminis. In fact, the pre-core gene encodes two forms of the same protein: the HBeAg extracellular form and the P22 or P25 intracellular forms hereby referred to as P22. The pre-core gene expression products are referred to as HBeAg/P22. The extracellular proteins are targets for immune mediated viral clearance mechanisms.

In relation to precore protein, translation is initiated from the first AUG of this ORF giving rise to a 25 kD polypeptide with the preC region encoding a signal peptide. The signal peptide functions by inserting the precursor protein into the ER where the peptide is cleaved resulting in a 17 kD protein product that is exported through the secretory pathway. The 17-25 kD protein is P22. During export, the basic C-terminal domain is cleaved off to generate a 15-17 kD soluble protein which is ultimately secreted into the serum and measured as HBeAg, but some is also incorporated into the outer cell membrane. HBeAg/P22 is not required for productive viral replication.

HBcAg is a 21 kD phosphoprotein whose synthesis is initiated from the second in-frame initiation codon and is translated from the shorter of the genomic transcripts. The HBcAg is the major protein component of the nucleocapsid. The C-terminal domain is highly basic and possesses a non-sequence specific nucleic acid binding domain.

The basal core promoter [BCP] (nucleotides 1744 to 1804), residing in the overlapping X open reading frame region (X-ORF), controls transcription of both pre-core and core regions and directs the synthesis of two mRNAs, the pre-core mRNA and the pre-genomic/C mRNA. The pre-core mRNA encodes HBeAg/P22 and the pre-genomic/C mRNA encodes the core protein. The DNA polymerase acts as the pre-genomic RNA (pgRNA) the template for reverse transcription.

The two major groups of mutations which affect HBeAg/P22 synthesis are pre-core protein mutations (G1896A) and mutations in the basal core promoter (BCP) at nucleotide 1762 and nucleotide 1764, all resulting in diminished production of HBeAg/P22 and a resultant increased host immune response although this may be transient in some patients and does not have a clear correlation with more aggressive liver disease in patients who are not immunosuppressed. Pre-core mutations frequently occur at a similar time and are often related to core gene mutations/deletions.

There is a relationship between the pre-core stop mutation and HBV genotypes. Nucleotide 1896 is a guanosine (G) and is found within the RNA structural element, epsilon, which is involved in encapsidation. This is base paired with nucleotide 1858 and mutations at nucleotide 1858, in conjunction with the pre-core stop mutation at nucleotide 1896 can enhance viral base pairing within the stem-loop region of epsilon. In patients with genotype A HBV infections (the most common genotype in North America and parts of Europe), nucleotide 1858 is a C. In this genotype, both a mutation at nucleotide 1896 (G to A) and nucleotide 1858 (C to T) would be required to stabilize the stem-loop structure. Without a compensatory mutation at nucleotide 1858 in genotype A HBV, impaired base pairing results when C-1858 tries to pair with a A-1896, which destabilizes the stem-loop structure of the packaging signal. Without a stable epsilon for packaging, decreased encapsidation and consequently decreased replication may occur resulting in a replication-deficient virus. Thus, pre-core stop codon mutations may be less frequent in genotype A because of the requirement for two mutational events. In contrast, HBV sequences in more than 70% of chronic HBV carriers from Asia, Africa, the Mediterranean basin or the Middle East already contain a T at nucleotide 1858. Thus, only a single mutation at nucleotide 1896 is required to yield a pre-core mutant with stable stem-loop pairing. This higher frequency of pre-core stop mutant HBV in patients harboring these other genotypes (genotypes B, C, D and E) where nucleotide 1858 is a T, is a reflection of the requirement of only a single mutation (G1896A) needed to cause a stop codon and a stable stem-loop structure for epsilon (Hunt et al, Hepatology. 31(5):1037-44, 1994; Lok et al, Proc Natl Acad Sci USA. 91(9):4077-81, 1994).

Mutations in the BCP, especially at nucleotide 1762 and nucleotide 1764, resulting in T-1762 and/or A-1764, have been detected in a variety of patients with persistent infection, fulminant hepatitis, as well as in immunosuppressed patients. The double mutation at T-1762 and A-1764 is associated with a decrease in HBeAg/P22 (but not disappearance) and an increase in viral load (Gunther et al, Adv Virus Res. 52:25-137, 1999; Hunt et al, 1994 supra). In general, this pattern of pre-core change is found in some genotype A infected patients.

The core protein can be divided into two major domains, the N-terminal assembly domain up to amino acid position 144 and the functionally important, arginine-rich C-terminal domain. The C-terminal domain is required for binding of the pre-genomic RNA and genome replication, as well as being involved in nuclear transportation. Interestingly, core protein sequences of HBV from patients in the HBeAg-positive immune tolerant phase contain none or very few amino acid changes suggesting that less immune pressure may result in less clinically evident mutations. The prevalence of HbcAg and HBeAg amino acid changes is very similar to that of pre-C defects and is seen during multiple stages of chronic infection. However, once patients enter the immune reactivation (clearance) phase, the mean rate of HbcAg and HBcAg amino acid changes increase by more than five-fold, clustering onto 36 hot-spot positions possibly influenced by the immune pressure and subsequent virus “selection”. These hot-spot positions have been linked to major cytotoxic T lymphocyte (CTL) [amino-acid 18-30] and T-helper (TH) cell [amino-acid 50-70] regions, and two B-cell [HBc/e1 and HBc/e2] epitopes at amino-acid residues 75-90 and 120-140 respectively (Gunther et al, 1999 supra).

To understand the special populations with chronic HBV infection one must understand the natural history of HBV infection. With up to 30% of patients with chronic HBV infection developing cirrhosis and or liver cancer, the course of HBV must be defined individually for each patient being evaluated for clinical trials or treatment. HBeAg negative chronic hepatitis currently represents the predominant form of chronic hepatitis due to HBV in several parts of the world where non genotype A infection is common eg., Africa, Asia, Middle-East, Mediterranean Basin and South America.

The important spontaneous seroconversion from HBeAg to anti-HBeAg antibodies (and a concommitant decrease in HBV DNA levels) occurs in 1 to 10% of chronic hepatitis B carriers (in patients with wild type virus) per annum, but seroconversion from HBsAg to anti-HBsAg antibodies with clearance of HBV from the liver, is very uncommon (at or less than 1% per year).

Chronic HBV infection is defined as the persistence of HBsAg for more than six months. HBV persistence may be due to the stable nature of covalently closed circular (cccDNA), infection of immunologically privileged sites and/or HBV-specific immune suppression. It is believed that HBeAg plays a role in HBV persistence by depleting HBeAg- and HBcAg-specific Th1 CD4+ T-cells via FAS-mediated apoptosis (Milich et al, J. Immunol 160:2013-21, 1998). HBeAg crosses the placenta and, therefore, may establish tolerance to HBV in newborns, increasing the frequency of persistent HBV infection with vertical transmission. The imbalance of Th1/Th2 responses promotes suppression of HBeAg-HBcAg-specific CD8+ T-cell responses and Th1 effector cells by production of anti-inflammatory cytokines such as IL-4 and IL-10 (Ferrari et al, J Immunol. 145:3442-9, 1990; Milich et al, 1998 supra, Milich et al, Proc Natl Acad Sci USA. 92:6847-51, 1995). There are also other mechanisms that may cause a generalized CD4+ T-cell hyporesponsiveness in individuals with chronic HBV infection because responses to mitogens are decreased when compared to HBV-negative controls and increased after HBV viral load is reduced with anti-HBV therapy (Boni et al, J Clin Invest. 102:968-75, 1998). This T-cell “hyporesponsiveness” may arise from decreased function in HBV infected Dendritic cells, which have reduced IFN-γ, TFN-α and IL-12 production and hence reduced stimulation of CD8+ T-cell responses (Beckebaum et al, Immunology. 109:487-95, 2003).

Overall, there is a reduction in functional HBV-specific CD4+ and CD8+ T-cell in persistent HBV infection when compared with individuals who successfully clear infection. In individuals with persistent HBV infection, the HBV-specific CD8+ T-cell response is significantly diminished when evaluated by proliferative responses to whole HBV antigens or defined epitopes in HLA-A2 positive chronic carriers (Ferrari et al, J Immunol. 145:3442-9, 1990; Maini et al, J Exp Med. 191:1269-80, 2000). In particular, in HBeAg-positive chronic carriers, specific CD8+ T-cells that recognize the core epitope (in region c18-27) are almost undetectable when measured by tetramers, and have diminished ability to produce IFN-γ. HBV-specific CD8+ T-cells are also found in the liver where they may cause an inflammatory response but are ineffective in clearing HBV infection (Jung et al, Virology. 261:165-72, 1999; Maini et al, 2000 supra).

The host virus relationship is a dynamic process in which many viruses such as HBV attempt to maximize their invisibility while the host attempts to prevent and eradicate infection. Initially, a virus must bind and enter a target cell and migrate to the appropriate cellular compartment in order to replicate and infect other cells. Infected cells may be triggered by the virus to produce cyokines (e.g. TNF-α and IFN-γ) that inhibit one or more stages of the viral replication cycle, thereby limiting the extent of the infection.

Host monocytes and macrophages play a key role in the early response to the virus as they secrete pro-inflammatory cytokines, such as IL-1, TNF-α, IL-6, IL-12 and IL-18 that have indirect and direct effects on the infection. They can recruit further monocytes, natural killer (NK) cells and T-cells to perform functions and they can also help switch to the appropriate Th function to help eradicate the virus.

Innate immunity to microbial pathogens, leading to the production of these pro-inflammatory cytokines, occurs as a result of the activation of Toll Like Receptors (TLRs). TLRs have been identified as a major class of pattern-recognition receptors. The role of TLRs involving bacterial products, e.g. endotoxin and peptidoglycan has recently been clarified (Akashi et al., J Immunol. 164: 3471-3475, 2000; Takeuchi et al., Immunity, 11: 443-451, 1999; Tapping et al., J Immunol. 165: 5780-5787, 2000). More than 13 TLRs have been identified and they play an important role in activation by a number of different bacteria. Recently, this has been extended to viruses with the demonstration that respiratory syncytial virus (RSV) stimulates TLR-4 in a murine model (Kurt-Jones et al., Nat Immunol. 1: 398-401, 2000; Haeberle et al., J Infect Dis. 186: 1199-1206, 2002). In addition, Measles Virus (MV) has been shown to activate TLR-2 dependent signals (Bieback et al., J Virol. 76: 8729-8736, 2002) and double-stranded RNA (the core of many viruses) has been shown to directly mediate responses to through TLR-3 (Matsumoto et al., Biochem Biophys Res Commun. 293: 1364-1369, 2002).

Stimulation of TLRs by their ligands initiates the activation of complex networks of intracellular signal transduction pathways to coordinate the ensuing inflammatory response. Important components of these signalling networks are the adaptor protein MyD88 (and related proteins), several protein kinases (including IRAK-1, p38 MAP kinase and IκB kinase), TRAF6 and the transcription factor NF-κB (FIG. 7). Activation of NF-κB leads to the expression of a variety of pro-inflammatory mediators (e.g. TNFα, IL-1, IL-6 and MCP-1) (Akira, S. J Biol Chem 278, 38105-8; 2003; Barton, G. M. & Medzhitov, R. Science 300, 1524-5 2003; Beutler, B., et al., J Leukoc Biol 74, 479-85; 2003). TLR3 and TLR4 are also capable of signalling via MyD88-independent pathways, involving the adaptor molecules TRIF (for TLR3 and 4) and TRAM (for TLR4) Lien, E. & Golenbock, D. T. Nat Immunol 4, 1162-4, 2003.

The signals induced upon TLR activation in turn control the activation of the specific immune response. There is evidence that the specific immune system only responds to a pathogen after it has been recognized and processed by the innate immune system. T-cell receptors require co-stimulatory molecules, such as CD80 and CD86, to be expressed on the surface of the antigen-presenting cell in association with the peptide-MHC complex in order for activation to occur. The expression of these co-stimulatory molecules is controlled in part by TLRs (Pasare, C. & Medzhitov, R. Curr Opin Immunol 15, 677-82, 2003). They are also important in activating B-cells to produce rheumatoid factors (Leadbetter, E. A. et al. Nature 416, 603-7 2002).

There is a need to investigate the role of pathogen-medicated down-regulation of TLRs and to develop mechanisms to combat infection by assisting the innate immunity system.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention identifies cell surface markers whose expression or activity are modified by the presence or absence of an HBV-specified effector molecule. It is proposed that the cell surface markers are involved in innate immunity and HBV-directed molecules specifically modulate the level or activity of these markers. The cell surface markers and the HBV-specified effector molecules are, therefore, useful therapeutic and/or diagnostic targets. In particular, the present invention identifies a modulation of Toll-like receptors (TLRs) in the presence of a HBV-specified antigen and hence both the antigen and the TLRs are useful therapeutic and diagnostic markers for HBV infection and to monitor treatment protocols. In an even more particular embodiment, the HBV-specified effector molecule is pre-core protein such as the intracellular form P22 or P25 or a secreted form thereof such as HBeAg. Collectively, these molecules are referred to herein as “HBeAg/P22”.

In accordance with a preferred embodiment of the present invention, it is identified that TLRs and in particular TLR-2 and TLR-4 are differentially affected by the presence or absence of HBeAg/P22 on or in liver cells following infection by HBV or a mutant form thereof. A mutant form of HBV includes the pre-core mutant and/or the BCP mutant. The pre-core protein (P22) or a secreted form thereof (HBeAg) and TLRs, and in particular, TLR-2 and TLR-4, are, therefore, useful targets for therapeutic or prophylactic agents including vaccines to treat or help prevent infection by HBV. They are also useful diagnostic targets to determine whether a subject is or has been infected by HBV or whether the subject is predisposed to or has a persistent infection or has another disease condition and can be used as a clinical or epidemiological management tool.

In particular, infection by HBV results in down-regulation of the TLRs which facilitates the infection process. Infection by a mutant HBV such as a pre-core mutant results in up-regulation of the TLRs.

The present invention provides, therefore, therapeutic and/or prophylactic agents capable of modulating levels of an HBV-specified effector molecule such as HBeAg/P22 and/or a TLR, such as TLR-2 and TLR-4. An HBV-specified effector molecule includes any molecule which up-regulates or down-regulates (i.e. modulates) a TLR such as TLR-2 or TLR-4. In HBV, for example, the effector molecule is HBeAg/P22.

The present invention further provides a method for detecting the presence of infection by HBV or a disease condition or a predisposition thereto, said method comprising determining the presence or absence of an HBV-specified effector molecule which modulates the level of TLR signalling wherein the presence or absence of the effector molecule or an elevated or reduced level of the TLR or a component within the TLR signalling pathway is indicative of infection by HBV or the presence of an associated disease condition or predisposition thereto.

The present invention further provides methods for diagnosis or assessment of infection by a HBV by the presence and/or levels of the HBV-specified effector molecule which modulates levels of a TLR or determining levels of TLRs such as TLR-2 and/or TLR-4 on liver cells.

The present invention also provides methods for diagnosis or assessment of infection by HBV by determining the presence and/or levels of the HBV-specified effector molecule which modulates levels of TLR signalling or determining levels of TLRs and components of the signalling pathway such as TLR-2 and/or TLR-4 and NFκβ on liver cells.

The present invention contemplates, therefore, therapeutic and diagnostic agents and compositions comprising same useful in the treatment, prophylaxis and/or diagnosis of infection by HBV or a mutant thereof or a predisposition to, or persistence, or clearance of infection. This aspect of the present invention particularly extends to the treatment and diagnosis of HBV infection and distinguishing between infection by an HBV with or without a pre-core mutation.

The present invention also provides a method of treating a subject infected with HBV or having a disease condition or having a predisposition thereto, said method comprising administering to said subject an effective amount of an agent including a vaccine which down-regulates the level of intracellular or extracellular pre-core expression product or up- or down-regulates the level of a TLR.

The present invention further provides a method for monitoring a response to therapy as well as determining the efficacy of a therapeutic regimen.

In addition, the present invention contemplates a method for monitoring a response to a therapeutic protocol directed against infection by HBV or development of a disease condition said method comprising determining the level or activity of a HBV-specified effector molecule which modulates TLR signalling wherein the presence or absence of the effector molecule or an elevated or reduced level of the TLR or a component within the TLR signalling pathway is indicative of infection by HBV or the presence of an associated disease condition or predisposition thereto.

Preferred mammals are humans. Animal models are also contemplated by the present invention.

### BRIEF DESCRIPTION OF THE FIGURES

FIG. 1 is a graphical representation showing levels of TLR-2 and TLR-4 in HBV wildtype infected cells compared to pre-core mutant HBV infected cells. A baculovirus system was employed.

FIG. 2 is a graphical representation showing levels of TLR-2 and TLR-4 in HBV wildtype infected cells compared to pre-core mutant HBV infected cells. A baculovirus system was employed with 100 moi.

FIG. 3 is a graphical representation of levels of TLR2 on liver cells. Top Panel: Single cell suspension flow cytometry histograms of hepatocyte TLR2 and TLR4 from hepatic biopsies of an example patient with steatosis (dashed line), patient with chronic HBV (shaded) and isotype control (dotted line). This demonstrates the TLR2 down-regulation on the surface of the hepatocytes in a patient with chroninc HBV as compared to an individual with the normal liver biopsy.

Lower Panels Peripheral blood and hepatic biopsies from three patients with HBeAg-positive HBV infection, five patients with HBeAg-negative HBV infection and five steatosis controls (C) were examined. Peripheral blood (blood) was analysed for TLR2 and TLR4 on CD14 positive monocytes. Liver biopsy tissue was separated into a single cell suspension and run on the flow cytometer. Kupffer cells and Hepatocytes were then gated and TLR2 and TLR4 were measured. The geometric mean fluorescence was expressed as a ratio to that of an isotype control antibody. The data is presented as % change in TLR. Mean and standard deviations from the individual experiments are shown. Asterisk indicate p values <0.05 compared with controls.

FIG. 4 is a graphical representation of the change in TLR2, TLR4 and TNF-α levels. Heparinised whole blood was stimulated for 20 hours with varying doses of HBV virus and TLR2 levels (B) and TLR4 (A) were measured on CD14 positive monocytes. The geometric mean fluorescence was expressed as a ratio to that of an isotype control antibody. The data is presented as % change in TLR. Panel (C) represents the TNF-α in the supernatant as measured by ELISA. The mean and standard deviations of 5 individual experiments from different donors are shown. The results for TLR2 were significant with a p<0.02, as was the TNF results with a p<0.02.

FIG. 5 is a graphical representation of changes in TLR levels and in TNF-α levels. Heparinised whole blood was stimulated for 20 hours with medium (C), wild type 1×10⁷ virus particles of HBV (WT), Hepatitis B Surface antigen (sAg), HBV Pre-core protein (PC) and HBV Core protein (Co). TLR2 and TLR4 levels were measured on CD14 positive monocytes (upper panel). The geometric mean fluorescence was expressed as a ratio to that of an isotype control antibody. The bottom panel represents the TNF-α in the supernatant as measured by ELISA for the stimulations demonstrated above. In addition two different genotypes of HBV (A and D) are demonstrated. The data are presented as % change in TLR. The mean and standard deviation of 5 individual experiments from different donors are shown. Asterisk indicate p values <0.05 compared with controls.

FIG. 6 is a graphical representation showing change in TLR and TNF levels. Mock infected (M), pre-core (PC) and core (C) baculovirus constructs in HepG2 cells were run on the flow cytometer and TLR2 and TLR4 was measured. The geometric mean fluorescence was expressed as a ratio to that of an isotype control antibody (top panel). The data are presented as % change in TLR. The bottom panel represents the same cells subjected to TaqMan Real time PCR (QPCR). This was performed in 384-well plate using the Assays-On-Demand Gene Expression Products (Applied Biosystems) and an ABI Prism 7900HT Sequence Detection System (Applied Biosystems). The relative amounts of PCR product were determined using the comparative Ct method, where the amount of target DNA was normalised to 18s and relative to the mock cDNA (2-deltadeltaCT). Recombinant HBV baculovirus constructs were generated by site-directed mutagenesis and co-transfection, using a 1.3 genome length wildtype (WT) HBV template (genotype D, subtype ayw) (Invitrogen, Stratagene, Calif.), as previously described. HepG2 cells were then transduced in parallel with WT, PC, BCP, and PC/BCP recombinant HBV baculovirus at a multiplicity of infection (MOI) of 50 plaque forming units (PFU) per cell. Fetal calf serum-free MEM was then used to make a single cell suspension, before staining for flow cytometry. Data represents the mean and standard deviation of three experiments. Asterisk indicate p values <0.05 compared with controls as determined by the non-parametric Mann Whitney-U test.

FIG. 7 is a diagrammatic representation of the TLR signalling pathway.

### DETAILED DESCRIPTION OF THE PREFERRED EMBODIMENTS

The present invention is predicated in part on the determination that infection by an HBV which produces a pre-core protein or a secreted form thereof such as HBeAg results in reduced levels of TLR-2 and TLR-4 in liver cells (including hepatocytes and Kupffer cells) and PBMCs (including peripheral monocytes) whereas infection by an HBV carrying a pre-core mutation and/or BCP mutation results in an up-regulation of TLR-2 and TLR-4 and may also modulate TLR signalling. The modulation of levels of TLR-2 and TLR-4 is determined by the HBV-specified effector molecule, pre-core protein or a secreted form thereof such as HBeAg. The presence, absence or levels of pre-core protein or a secreted form thereof such as HBeAg or levels of TLR-2 and TLR-4 and/or the function or activity of pre-core protein or a secreted form thereof such as HBeAg provide a diagnostic indicator of HBV infection or the type of HBV causing the infection or a predisposition to or persistence of HBV infection. Additionally, pre-core protein or a secreted form thereof such as HBeAg, TLR-2 and TLR-4 become therapeutic targets for agents including vaccines which modulate pre-core protein or a secreted form thereof such as HBeAg or TLR-2 and/or TLR-4 levels or components of the TLR signalling pathway.

The pre-core protein or a secreted form thereof such as HBeAg is hereinafter referred to as “pre-core protein/HBeAg”. The present invention extends to HBV variants carrying a pre-core mutatin such as a truncation, point or null mutation, and also the HBV variants with a BCP mutation(s) that down regulate transcription of the HBV precore gene.

The present invention provides, therefore, agents which modulate levels of pre-core protein/HBeAg and/or TLRs and in particular TLR-2 and/or TLR-4, or components of the TLR signalling pathway, diagnostic agents to determine the levels of pre-core protein/HBeAg and/or TLR-2 and/or TLR-4 and/or components of the TLR signalling pathway and methods for the treatment and/or prophylaxis of infection including development of a vaccine for HBV infection.

The present invention further contemplates a method for monitoring a response to a therapeutic protocol as well as a means for determining the efficacy of a therapeutic regimen. In particular, the present invention provides a clinical or epidemiological management tool for infection and development of other disease conditions in animals such as mammals and in particular humans.

The present invention further permits a distinction between an infection with an HBV which produces a pre-core protein/HBeAg or a virus which does not (ie., prcore and/or BCP mutant) based on the TLR levels or components of the TLR signalling pathway.

Accordingly, one aspect of the present invention contemplates a method for detecting the presence of infection by HBV or a disease condition or a predisposition thereto, said method comprising determining the presence or absence of an HBV-specified effector molecule which modulates the level or activity of a TLR or determining the level or activity of the TLR or a homolog thereof wherein the presence or absence of the effector molecule or an elevated or reduced level of the TLR or a homolog thereof is indicative of infection by the HBV or the presence of an associated disease condition or predisposition thereto.

In particular, the present invention provides a method for detecting the presence of infection by HBV or a disease condition or a predisposition thereto, said method comprising determining the presence or absence of an HBV-specified effector molecule which modulates the level of TLR signalling wherein the presence or absence of the effector molecule or an elevated or reduced level of the TLR or a component within the TLR signalling pathway is indicative of infection by HBV or the presence of an associated disease condition or predisposition thereto.

Furthermore, another aspect of the present invention contemplates a method for detecting the presence of infection by HBV or a disease condition or a predisposition thereto, said method comprising determining the presence or absence of an HBV-specified effector molecule which modulates the level or activity of a TLR or determining the level or activity of the TLR, or a homolog thereof, or components of the TLR signalling pathway wherein the presence or absence of the effector molecule or an elevated or reduced level of the TLR or a homolog or components of the TLR signalling pathway thereof is indicative of infection by the HBV or the presence of an associated disease condition or predisposition thereto.

Another embodiment of the present invention provides a method for monitoring a response to a therapeutic protocol directed against infection by HBV or development of a disease condition said method comprising determining the level or activity of a HBV-specified effector molecule or the level or activity of a TLR or a homolog thereof wherein the presence or absence of the effector molecule or an elevated or reduced level of the TLR or a homolog thereof, components of the TLR signalling pathway is indicative of infection by HBV or the presence of an associated disease condition or predisposition thereto.

Still yet another aspect of the present invention is directed to method of treating a subject infected with HBV or having a disease condition or having a predisposition thereto, said method comprising administering to said subject an effective amount of an agent including a vaccine which down-regulates pre-core protein/HBeAg or up- or down-regulates the level of a TLR or components of the signalling pathway.

Before describing the present invention in detail, it is to be understood that unless otherwise indicated, the subject invention is not limited to specific formulations of components, manufacturing methods, dosage regimens, or the like, as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context clearly dictates otherwise. Thus, for example, reference to a “compound” includes a single compound, as well as two or more compounds; reference to “an active agent” includes a single active agent, as well as two or more active agents; and so forth.

In describing and claiming the present invention, the following terminology are used in accordance with the definitions set forth below.

The terms “compound”, “active agent”, “pharmacologically active agent”, “medicament”, “active” and “drug” are used interchangeably herein to refer to a chemical compound that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. The term “compound” is not to be construed as a chemical compound only but extends to peptides, polypeptides and proteins as well as genetic molecules such as RNA, DNA and chemical analogs thereof. Reference to a “peptide”, “polypeptide” or “protein” includes molecules with a polysaccharide or lipopolysaccharide component. The term “antagonist” is an example of a compound, active agent, pharmacologically active agent, medicament, active and drug which down-regulates the level of pre-core protein/HBeAg or other pathogen-specific effector molecule or which down-regulates a TLR. An “agonist” or “potentiator” up-regulates the levels of pre-core protein/HBeAg or a TLR, such as TLR-2 or TLR-4.

The present invention extends to a vaccine and to combinbations of compounds or agents such as an agonist or antagonists of TLR-2 and/or 4 and a nucleoside analog or anti-pre-core/HBeAg antibody, or antiviral cytokines (eg., IFN-∝., IFN-γ, IL-2, TNF-∝) or other immunomodulatory agents.

The present invention contemplates, therefore, compounds useful in modulating levels of pre-core protein/HBeAg or a TLR such as TLR-2 and/or TLR-4 or potentiating general or specific TLR signaling. The compounds have an effect on reducing or preventing or treating infection by HBV or treating another disease condition. The preferred cells which carry the TLRs to be modulated include liver cells. A liver cell includes a hepatocyte. Reference to a “compound”, “active agent”, “pharmacologically active agent”, “medicament”, “active” and “drug” includes combinations of two or more actives such as an antagonist of pre-core protein/HBeAg or agonist or potentiator of TLR or TLR signaling. A “combination” also includes multi-part such as a two-part pharmaceutical composition where the agents are provided separately and given or dispensed separately or admixed together prior to dispensation.

For example, a multi-part pharmaceutical pack may have a modulator of pre-core protein/HBeAg or a TLR and one or more anti-microbial or anti-viral agents. The terms “modulating” or its derivatives, such as “modulate” or “modulation”, are used to describe up- or down-regulation.

The terms “effective amount” and “therapeutically effective amount” of an agent as used herein mean a sufficient amount of the agent to provide the desired therapeutic or physiological effect. Furthermore, an “effective HBeAg-modulating or TLR-modulating amount” of an agent is a sufficient amount of the agent to directly or indirectly up- or down-regulate the function of pre-core protein/HBeAg or up- or down-regulate a specific TLR such a TLR-2 or TLR-4 or to potentiate TLR signaling. This may be accomplished, for example, by the agents acting as an antagonist of pre-core protein/HBeAg or an agonist (i.e. a potentiator) of the TLR or its signaling components such as agents which are or mimic components of the TLR signaling pathway, by agents which induce the TLR signaling pathway via other cellular receptors or by the agents antagonizing inhibitors of TLR signaling components. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like. A pharmaceutical composition may also be described depending on the formulation as a vaccine composition.

Similarly, a “pharmacologically acceptable” salt, ester, emide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms of infection or disease, elimination of symptoms and/or underlying cause, prevention of the occurrence of symptoms of infection and/or their underlying cause and improvement or remediation of damage. Collateral damage, for example, following viral infection may be liver damage such as cirrhosis or hepatocellular carcinoma of the liver.

“Treating” a patient may involve prevention of infection or other disease condition or adverse physiological event in a susceptible individual as well as treatment of a clinically symptomatic individual by inhibiting an infection or other disease condition or downstream condition such as liver damage or cancer. Generally, such a condition or disorder is an infection, more particularly, a viral infection and, even more particularly, infection by HBV. Thus, for example, the subject method of “treating” a patient with an infection or with a propensity for one to develop encompasses both prevention of the infection or other disease condition as well as treating the infection or other disease condition once established.

Reference to “HBV” or its full term “Hepatitis B virus” includes all variants including variants resistant to particular therapeutic agents such as nucleoside analogs or immunological agents. Particularly important variants are pre-core and/or BCP mutants of HBV.

“Patient” as used herein refers to an animal, preferably a mammal and more preferably human who can benefit from the pharmaceutical formulations and methods of the present invention. There is no limitation on the type of animal that could benefit from the presently described pharmaceutical formulations and methods. A patient regardless of whether a human or non-human animal may be referred to as an individual, subject, animal, host or recipient. The compounds and methods of the present invention have applications in human medicine, veterinary medicine as well as in general, domestic or wild animal husbandry.

The compounds of the present invention may be large or small molecules, nucleic acid molecules (including antisense or sense molecules), peptides, polypeptides or proteins or hybrid molecules such as RNAi- or siRNA-complexes (including RISC complexes and Dicer complexes), ribozymes or DNAzymes. The compounds may need to be modified so as to facilitate entry into a cell. This is not a requirement if the compound interacts with an extracellular receptor. Examples of agents include chemical agents and antibodies which interact with pre-core protein/HBeAg or the TLR or genetic molecules which modulates pre-core expression.

As indicated above, the preferred animals are humans.

Examples of laboratory test animals (including animal modeling) include mice, rats, rabbits, guinea pigs and hamsters. Rabbits and rodent animals, such as rats and mice, provide a convenient test system or animal model. Livestock animals include sheep, cows, pigs, goats, horses and donkeys. Non-mammalian animals such as avian species (such as ducks), zebrafish, amphibians (including cane toads) and Drosophila species such as Drosophila melanogaster are also contemplated.

The present invention provides, therefore, agents which modulate (e.g agonize or antagonize) pre-core protein/HBeAg or modulate (i.e. potentiate or activate or antagonize) TLRs such as TLR-2 and/or TLR-4.

The present invention contemplates methods of screening for such agents comprising, for example, contacting a candidate drug with pre-core protein/HBeAg or a TLR such as TLR-2 or TLR-4 or a part thereof. The pre-core protein/HBeAg or TLR molecule is referred to herein as a “target” or “target molecule”. The screening procedure includes assaying (i) for the presence of a complex between the drug and the target, or (ii) an alteration in the expression levels of nucleic acid molecules encoding the target. One form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the compound rather than the target and to measure the amount of compound binding to target in the presence and in the absence of the drug being tested. Such compounds may inhibit the target which is useful, for example, in finding modulators of pre-core protein/HBeAg or modulators of a TLR required for the treatment or prophylaxis of HBV infection.

Another technique for drug screening provides high throughput screening for compounds having suitable binding affinity to a target and is described in detail in Geysen (International Patent Publication No. WO 84/03564). Briefly stated, large numbers of different small peptide test compounds are synthesized on a solid substrate, such as plastic pins or some other surface. The peptide test compounds are reacted with a target and washed. Bound target molecule is then detected by methods well known in the art. This method may be adapted for screening for non-peptide, chemical entities. This aspect, therefore, extends to combinatorial approaches to screening for target modulators of pre-core protein/HBeAg or of TLRs such as TLR-2 or TLR-4.

Purified target can be coated directly onto plates for use in the aforementioned drug screening techniques. However, non-neutralizing antibodies to the target may also be used to immobilize the target on the solid phase. Antibodies specific for pre-core protein/HBeAg may also be useful as inhibitors of pre-core protein/HBeAg.

The present invention also contemplates the use of competitive drug screening assays in which neutralizing antibodies capable of specifically binding the target compete with a test compound for binding to the target or fragments thereof. In this manner, the antibodies can be used to detect the presence of any peptide which shares one or more antigenic determinants of the target.

Antibodies to pre-core protein/HBeAg or a TLR may be polyclonal or monoclonal although monoclonal antibodies are preferred. Antibodies may be prepared by any of a number of means. For the detection of pre-core protein/HBeAg or a TLR, antibodies are generally but not necessarily derived from non-human animals such as primates, livestock animals (e.g. sheep, cows, pigs, goats, horses), laboratory test animals (e.g. mice, rats, guinea pigs, rabbits) and companion animals (e.g. dogs, cats). Generally, antibody based assays are conducted in vitro on cell or tissue biopsies. However, if an antibody is suitably deimmunized or, in the case of human use, humanized, then the antibody can be labeled with, for example, a nuclear tag, administered to a subject and the site of nuclear label accumulation determined by radiological techniques. The pre-core protein/HBeAg or TLR antibody is regarded, therefore, as a pathogenic marker targeting agent. Accordingly, the present invention extends to deimmunized forms of the antibodies for use in pathogenic target imaging in human and non-human subjects. This is described further below.

For the generation of antibodies to pre-core protein/HBeAg or a TLR, the molecule is required to be extracted from a biological sample whether this be from animal including human tissue or from cell culture if produced by recombinant means. Generally, monocytes and hepatocytes are a convenient source. The pre-core protein/HBeAg or TLR can be separated from the biological sample by any suitable means. For example, the separation may take advantage of any one or more of pre-core protein/HBeAg's or TLR's surface charge properties, size, density, biological activity and its affinity for another entity (e.g. another protein or chemical compound to which it binds or otherwise associates). Thus, for example, separation of pre-core protein/HBeAg or TLR from the biological sample may be achieved by any one or more of ultra-centrifugation, ion-exchange chromatography (e.g. anion exchange chromatography, cation exchange chromatography), electrophoresis (e.g. polyacrylamide gel electrophoresis, isoelectric focussing), size separation (e.g., gel filtration, ultra-filtration) and affinity-mediated separation (e.g. immunoaffinity separation including, but not limited to, magnetic bead separation such as Dynabead® separation, immunochromatography, immuno-precipitation). Choice of the separation technique(s) employed may depend on the biological activity or physical properties of the pre-core protein/HBeAg or particular TLR sought or from which tissues it is obtained.

Preferably, the separation of pre-core protein/HBeAg or the TLR from the biological fluid preserves conformational epitopes present on the kinase and, thus, suitably avoids techniques that cause denaturation of the molecule. Persons of skill in the art will recognize the importance of maintaining or mimicking as close as possible physiological conditions peculiar to pre-core protein/HBeAg or the TLR (e.g. the biological sample from which it is obtained) to ensure that the antigenic determinants or active site/s on the pre-core protein/HBeAg or TLR, which are exposed to the animal, are structurally identical to that of the native molecule. This ensures the raising of appropriate antibodies in the immunized animal that would recognize the native molecule.

Immunization and subsequent production of monoclonal antibodies can be carried out using standard protocols as for example described in Kohler and Milstein, Nature. 256: 495-499, 1975; Kohler and Milstein, Eur. J. Immunol. 6(7): 511-519, 1976), Coligan et al. (“Current Protocols in Immunology, John Wiley & Sons, Inc., 1991-1997) and Toyama et al. (Monoclonal Antibody, Experiment Manual”, published by Kodansha Scientific, 1987. Essentially, an animal is immunized with an pre-core protein/HBeAg or a TLR or a sample comprising an pre-core protein/HBeAg or a TLR by standard methods to produce antibody-producing cells, particularly antibody-producing somatic cells (e.g. B lymphocytes). These cells can then be removed from the immunized animal for immortalization.

Where a fragment of pre-core protein/HBeAg or TLR is used to generate antibodies, it may need to first be associated with a carrier. By “carrier” is meant any substance of typically high molecular weight to which a non- or poorly immunogenic substance (e.g. a hapten) is naturally or artificially linked to enhance its immunogenicity.

Immortalization of antibody-producing cells may be carried out using methods which are well-known in the art. For example, the immortalization may be achieved by the transformation method using Epstein-Barr virus (EBV) (Kozbor et al., Methods in Enzymology. 121: 140, 1986). In a preferred embodiment, antibody-producing cells are immortalized using the cell fusion method (described in Coligan et al., 1991-1997, supra), which is widely employed for the production of monoclonal antibodies. In this method, somatic antibody-producing cells with the potential to produce antibodies, particularly B cells, are fused with a myeloma cell line. These somatic cells may be derived from the lymph nodes, spleens and peripheral blood of primed animals, preferably rodent animals such as mice and rats. Mice spleen cells are particularly useful. It would be possible, however, to use rat, rabbit, sheep or goat cells, or cells from other animal species instead.

Specialized myeloma cell lines have been developed from lymphocytic tumors for use in hybridoma-producing fusion procedures (Kohler and Milstein, 1976, supra; Shulman et al., Nature. 276: 269-270, 1978; Volk et al., J. Virol. 42(1): 220-227, 1982). These cell lines have been developed for at least three reasons. The first is to facilitate the selection of fused hybridomas from unfused and similarly indefinitely self-propagating myeloma cells. Usually, this is accomplished by using myelomas with enzyme deficiencies that render them incapable of growing in certain selective media that support the growth of hybridomas. The second reason arises from the inherent ability of lymphocytic tumor cells to produce their own antibodies. To eliminate the production of tumor cell antibodies by the hybridomas, myeloma cell lines incapable of producing endogenous light or heavy immunoglobulin chains are used. A third reason for selection of these cell lines is for their suitability and efficiency for fusion.

Many myeloma cell lines may be used for the production of fused cell hybrids, including, e.g. P3X63-Ag8, P3X63-AG8.653, P3/NS1-Ag4-1 (NS-1), Sp2/0-Ag14 and S194/5.XXO.Bu.1. The P3X63-Ag8 and NS-1 cell lines have been described by Köhler and Milstein (1976, supra). Shulman et al. (1978, supra) developed the Sp2/0-Ag14 myeloma line. The S194/5.XXO.Bu.1 line was reported by Trowbridge (J. Exp. Med. 148(1): 313-323, 1978).

Methods for generating hybrids of antibody-producing spleen or lymph node cells and myeloma cells usually involve mixing somatic cells with myeloma cells in a 10:1 proportion (although the proportion may vary from about 20:1 to about 1:1), respectively, in the presence of an agent or agents (chemical, viral or electrical) that promotes the fusion of cell membranes. Fusion methods have been described (Kohler and Milstein, 1975, supra; Kohler and Milstein, 1976, supra; Gefter et al., Somatic Cell Genet. 3: 231-236, 1977; Volk et al, 1982, supra). The fusion-promoting agents used by those investigators were Sendai virus and polyethylene glycol (PEG).

Because fusion procedures produce viable hybrids at very low frequency (e.g. when spleens are used as a source of somatic cells, only one hybrid is obtained for roughly every 1×10⁵ spleen cells), it is preferable to have a means of selecting the fused cell hybrids from the remaining unfused cells, particularly the unfused myeloma cells. A means of detecting the desired antibody-producing hybridomas among other resulting fused cell hybrids is also necessary. Generally, the selection of fused cell hybrids is accomplished by culturing the cells in media that support the growth of hybridomas but prevent the growth of the unfused myeloma cells, which normally would go on dividing indefinitely. The somatic cells used in the fusion do not maintain long-term viability in in vitro culture and hence do not pose a problem. In the example of the present invention, myeloma cells lacking hypoxanthine phosphoribosyl transferase (HPRT-negative) were used. Selection against these cells is made in hypoxanthine/aminopterin/thymidine (HAT) medium, a medium in which the fused cell hybrids survive due to the HPRT-positive genotype of the spleen cells. The use of myeloma cells with different genetic deficiencies (drug sensitivities, etc.) that can be selected against in media supporting the growth of genotypically competent hybrids is also possible.

Several weeks are required to selectively culture the fused cell hybrids. Early in this time period, it is necessary to identify those hybrids which produce the desired antibody, so that they may subsequently be cloned and propagated. Generally, around 10% of the hybrids obtained produce the desired antibody, although a range of from about 1 to about 30% is not uncommon. The detection of antibody-producing hybrids can be achieved by any one of several standard assay methods, including enzyme-linked immunoassay and radioimmunoassay techniques as, for example, described in Kennet et al. (Monoclonal Antibodies and Hybridomas: A New Dimension in Biological Analyses, pp 376-384, Plenum Press, New York, 1980) and by FACS analysis (O'Reilly et al., Biotechniques. 25: 824-830, 1998).

Once the desired fused cell hybrids have been selected and cloned into individual antibody-producing cell lines, each cell line may be propagated in either of two standard ways. A suspension of the hybridoma cells can be injected into a histocompatible animal. The injected animal will then develop tumors that secrete the specific monoclonal antibody produced by the fused cell hybrid. The body fluids of the animal, such as serum or ascites fluid, can be tapped to provide monoclonal antibodies in high concentration. Alternatively, the individual cell lines may be propagated in vitro in laboratory culture vessels. The culture medium containing high concentrations of a single specific monoclonal antibody can be harvested by decantation, filtration or centrifugation, and subsequently purified.

The cell lines are tested for their specificity to detect pre-core protein/HBeAg or the TLR of interest by any suitable immunodetection means. For example, cell lines can be aliquoted into a number of wells and incubated and the supernatant from each well is analyzed by enzyme-linked immunosorbent assay (ELISA), indirect fluorescent antibody technique, or the like. The cell line(s) producing a monoclonal antibody capable of recognizing the target pre-core protein/HBeAg or TLR but which does not recognize non-target epitopes are identified and then directly cultured in vitro or injected into a histocompatible animal to form tumors and to produce, collect and purify the required antibodies.

These antibodies are pre-core protein/HBeAg- or TLR-specific. This means that the antibodies are capable of distinguishing a particular pre-core protein/HBeAg or TLR from other molecules. More broad spectrum antibodies may be used provided that they do not cross-react with molecules in a normal cell.

Where the monoclonal antibody is destined for use as a therapeutic agent such as to inhibit pre-core protein/HBeAg, then, it will need to be deimmunized with respect to the host into which it will be introduced (e.g. a human). The deimmunization process may take any of a number of forms including the preparation of chimeric antibodies which have the same or similar specificity as the monoclonal antibodies prepared according to the present invention. Chimeric antibodies are antibodies whose light and heavy chain genes have been constructed, typically by genetic engineering, from immunoglobulin variable and constant region genes belonging to different species. Thus, in accordance with the present invention, once a hybridoma producing the desired monoclonal antibody is obtained, techniques are used to produce interspecific monoclonal antibodies wherein the binding region of one species is combined with a non-binding region of the antibody of another species (Liu et al., Proc. Natl. Acad. Sci. USA. 84: 3439-3443, 1987). For example, complementary determining regions (CDRs) from a non-human (e.g. murine) monoclonal antibody can be grafted onto a human antibody, thereby “humanizing” the murine antibody (European Patent No. 0 239 400; Jones et al., Nature. 321: 522-525, 1986; Verhoeyen et al., Science. 239: 1534-1536, 1988; Richmann et al., Nature. 332: 323-327, 1988). In this case, the deimmunizing process is specific for humans. More particularly, the CDRs can be grafted onto a human antibody variable region with or without human constant regions. The non-human antibody providing the CDRs is typically referred to as the “donor” and the human antibody providing the framework is typically referred to as the “acceptor”. Constant regions need not be present, but if they are, they must be substantially identical to human immunoglobulin constant regions, i.e. at least about 85-90%, preferably about 95% or more identical. Hence, all parts of a humanized antibody, except possibly the CDRs, are substantially identical to corresponding parts of natural human immunoglobulin sequences. Thus, a “humanized antibody” is an antibody comprising a humanized light chain and a humanized heavy chain immunoglobulin. A donor antibody is said to be “humanized”, by the process of “humanization”, because the resultant humanized antibody is expected to bind to the same antigen as the donor antibody that provides the CDRs. Reference herein to “humanized” includes reference to an antibody deimmunized to a particular host, in this case, a human host.

It will be understood that the deimmunized antibodies may have additional conservative amino acid substitutions which have substantially no effect on antigen binding or other immunoglobulin functions. Exemplary conservative substitutions may be made according to Table 1.

TABLE 1

| ORIGINAL | EXEMPLARY |
|---|---|
| RESIDUE | SUBSTITUTIONS |
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Exemplary methods which may be employed to produce deimmunized antibodies according to the present invention are described, for example, in Richmann et al., 1988, supra; European Patent No. 0 239 400; U.S. Pat. No. 6,056,957, U.S. Pat. No. 6,180,370, U.S. Pat. No. 6,180,377.

Thus, in one embodiment, the present invention contemplates a deimmunized antibody molecule having specificity for an epitope recognized by a monoclonal antibody to pre-core protein/HBeAg wherein at least one of the CDRs of the variable domain of said deimmunized antibody is derived from the said monoclonal antibody to said pre-core protein/HBeAg and the remaining immunoglobulin-derived parts of the deimmunized antibody molecule are derived from an immunoglobulin or an analog thereof from the host for which the antibody is to be deimmunized.

This aspect of the present invention involves manipulation of the framework region of a non-human antibody.

The present invention extends to mutants and derivatives of the subject antibodies but which still retain specificity for pre-core protein/HBeAg.

The terms “mutant” or “derivatives” includes one or more amino acid substitutions, additions and/or deletions.

As used herein, the term “CDR” includes CDR structural loops which covers to the three light chain and the three heavy chain regions in the variable portion of an antibody framework region which bridge β strands on the binding portion of the molecule. These loops have characteristic canonical structures (Chothia et al., J. Mol. Biol. 196: 901, 1987; Chothia et al., J. Mol. Biol. 227: 799, 1992).

By “framework region” is meant region of an immunoglobulin light or heavy chain variable region, which is interrupted by three hypervariable regions, also called CDRs. The extent of the framework region and CDRs have been precisely defined (see, for example, Kabat et al., “Sequences of Proteins of Immunological Interest”, U.S. Department of Health and Human Sciences, 1983). The sequences of the framework regions of different light or heavy chains are relatively conserved within a species. As used herein, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDRs. The CDRs are primarily responsible for binding to an epitope of the HBeAg.

As used herein, the term “heavy chain variable region” means a polypeptide which is from about 110 to 125 amino acid residues in length, the amino acid sequence of which corresponds to that of a heavy chain of a monoclonal antibody of the invention, starting from the amino-terminal (N-terminal) amino acid residue of the heavy chain. Likewise, the term “light chain variable region” means a polypeptide which is from about 95 to 130 amino acid residues in length, the amino acid sequence of which corresponds to that of a light chain of a monoclonal antibody of the invention, starting from the N-terminal amino acid residue of the light chain. Full-length immunoglobulin “light chains” (about 25 Kd or 214 amino acids) are encoded by a variable region gene at the NH₂-terminus (about 110 amino acids) and a κ or λ constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g. γ (encoding about 330 amino acids).

The term “immunoglobulin” or “antibody” is used herein to refer to a protein consisting of one or more polypeptides substantially encoded by immunoglobulin genes. The recognized immunoglobulin genes include the κ. λ, α. γ (IgG₁, IgG₂, IgG₃, IgG₄), δ. ε and μ constant region genes, as well as the myriad immunoglobulin variable region genes. One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions. In addition to antibodies, immunoglobulins may exist in a variety of other forms including, for example, Fv, Fab, Fab′ and (Fab′)₂.

The present invention also contemplates the use and generation of fragments of monoclonal antibodies produced by the method of the present invention including, for example, Fv, Fab, Fab′ and F(ab′)₂ fragments. Such fragments may be prepared by standard methods as for example described by Coligan et al. (1991-1997, supra).

The present invention also contemplates synthetic or recombinant antigen-binding molecules with the same or similar specificity as the monoclonal antibodies of the invention. Antigen-binding molecules of this type may comprise a synthetic stabilized Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Krebber et al. J. Immunol. Methods. 201(1): 35-55, 1997. Alternatively, they may be prepared by methods described in U.S. Pat. No. 5,091,513, European Patent No 239,400 or the articles by Winter and Milstein (Nature. 349: 293, 1991) and Plückthun et al. (In Antibody engineering: A practical approach, 203-252, 1996).

Alternatively, the synthetic stabilized Fv fragment comprises a disulphide stabilized Fv (dsFv) in which cysteine residues are introduced into the V_{H} and V_{L} domains such that in the fully folded Fv molecule the two residues will form a disulphide bond there between. Suitable methods of producing dsFv are described, for example, in (Glockshuber et al., Biochem. 29: 1363-1367, 1990; Reiter et al., J. Biol. Chem. 269: 18327-18331, 1994; Reiter et al., Biochem. 33: 5451-5459, 1994; Reiter et al., Cancer Res. 54: 2714-2718, 1994; Webber et al., Mol. Immunol. 32: 249-258, 1995).

Also contemplated as synthetic or recombinant antigen-binding molecules are single variable region domains (termed dAbs) as, for example, disclosed in (Ward et al., Nature. 341: 544-546, 1989; Hamers-Casterman et al., Nature. 363: 446-448, 1993; Davies and Riechmann, FEBS Lett. 339: 285-290, 1994).

Alternatively, the synthetic or recombinant antigen-binding molecule may comprise a “minibody”. In this regard, minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the synthetic or recombinant antigen binding molecule may comprise non-immunoglobulin derived, protein frameworks. For example, reference may be made to (Ku & Schutz, Proc. Natl. Acad. Sci. USA. 92: 6552-6556, 1995) which discloses a four-helix bundle protein cytochrome b562 having two loops randomized to create CDRs, which have been selected for antigen binding.

The synthetic or recombinant antigen-binding molecule may be multivalent (i.e. having more than one antigen binding site). Such multivalent molecules may be specific for one or more antigens. Multivalent molecules of this type may be prepared by dimerization of two antibody fragments through a cysteinyl-containing peptide as, for example disclosed by (Adams et al., Cancer Res. 53: 4026-4034, 1993; Cumber et al., J. Immunol. 149: 120-126, 1992). Alternatively, dimerization may be facilitated by fusion of the antibody fragments to amphiphilic helices that naturally dimerize (Plünckthun, Biochem 31: 1579-1584, 1992) or by use of domains (such as leucine zippers jun and fos) that preferentially heterodimerize (Kostelny et al., J. Immunol. 148: 1547-1553, 1992). Multivalent antibodies are useful, for example, in detecting different forms of TLRs such as TLR-2 and TLR-4.

Yet another useful source of compounds useful in modulating pre-core protein/HBeAg or TLR activity or levels is a chemically modified ligand of pre-core protein/HBeAg or the TLR.

In addition, compounds can be selected which interrupt or antagonize or agonize the interaction between pre-core protein/HBeAg and a TLR.

Analogs of proteinaceous molecules (e.g. ligands of pre-core protein/HBeAg or a TLR) contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 2.

TABLE 2
Codes for non-conventional amino acids

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl-carboxylate | Norb | L-N-methylglutamine | Nmgln |
| | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

Accordingly, one aspect of the present invention contemplates any compound which binds or otherwise interacts with pre-core protein/HBeAg or a TLR, such as TLR-2 or TLR-4, or a component of a TLR signaling pathway resulting in modulation of pre-core protein/HBeAg or TLR levels or activity.

Another useful group of compounds is a mimetic. The terms “peptide mimetic”, “target mimetic” or “mimetic” are intended to refer to a substance which has some chemical similarity to the target but which antagonizes or agonizes or mimics the target. The target in this case may be a ligand of pre-core protein/HBeAg or of the TLR. A peptide mimetic may be a peptide-containing molecule that mimics elements of protein secondary structure (Johnson et al., “Peptide Turn Mimetics” in Biotechnology and Pharmacy, Pezzuto et al., Eds., Chapman and Hall, New York, 1993). The underlying rationale behind the use of peptide mimetics is that the peptide backbone of proteins exists chiefly to orient amino acid side chains in such a way as to facilitate molecular interactions such as those of antibody and antigen, enzyme and substrate or scaffolding proteins. A peptide mimetic is designed to permit molecular interactions similar to the natural molecule. Peptide or non-peptide mimetics may be useful, for example, to competitively inhibit or otherwise bind to pre-core protein/HBeAg or to activate a TLR or TLR pathway. Preferred TLRs in this instance are TLR-2 and TLR-4.

Again, the compounds of the present invention may be selected to interact with a target alone or single or multiple compounds may be used to affect multiple targets. For example, multiple targets may include an pre-core protein/HBeAg and the pathogen itself. For example, one useful therapeutic combination would be an antgonist of pre-core protein/HBeAg and a nucleoside analog and/or antiviral cytokine

The target or fragment employed in screening assays may either be free in solution, affixed to a solid support, or borne on a cell surface. One method of drug screening utilizes eukaryotic or prokaryotic host cells which are stably transformed with recombinant polynucleotides expressing the pre-core protein/HBeAg or TLR or fragment, preferably in competitive binding assays. Such cells, either in viable or fixed form, can be used for standard binding assays. One may measure, for example, the formation of complexes between an pre-core protein/HBeAg or a TLR or fragment and the agent being tested, or examine the degree to which the formation of a complex between an pre-core protein/HBeAg or a TLR or fragment and a ligand is aided or interfered with by the agent being tested.

A substance identified as a modulator of target function or gene activity may be a peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. Alanine scans of peptides are commonly used to refine such peptide motifs. These parts or residues constituting the active region of the compound are known as its “pharmacophore”.

Once the pharmacophore has been found, its structure is modeled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of an pre-core protein/HBeAg or a TLR and their ligands are modeled. This can be especially useful where the pre-core protein/HBeAg or TLR and/or their ligands change conformation on binding, allowing the model to take account of this in the design of the mimetic. Modeling can be used to generate inhibitors which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, e.g. enhance or interfere with the function of a polypeptide in vivo. See, e.g. Hodgson (Bio/Technology. 9: 19-21, 1991). In one approach, one first determines the three-dimensional structure of an pre-core protein/HBeAg or a TLR ligand by x-ray crystallography, by computer modeling or most typically, by a combination of approaches. Useful information regarding the structure of an pre-core protein/HBeAg or a TLR ligand may also be gained by modeling based on the structure of homologous proteins. An example of rational drug design is the development of HIV protease inhibitors (Erickson et al., Science. 249: 527-533, 1990). In addition, target molecules may be analyzed by an alanine scan (Wells, Methods Enzymol. 202: 2699-2705, 1991). In this technique, an amino acid residue is replaced by Ala and its effect on the peptide's activity is determined. Each of the amino acid residues of the peptide is analyzed in this manner to determine the important regions of the peptide.

It is also possible to isolate an pre-core protein/HBeAg-specific or TLR-specific antibody (such as by the method described above) and then to solve its crystal structure. In principle, this approach yields a pharmacophore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacophore.

The present invention extends to a genetic approach to up-regulating or down-regulating expression of a gene encoding a pre-core protein/HBeAg or a TLR, such as TLR-2 or TLR-4. Generally, it is more convenient to use genetic means to induce gene silencing such as pre- or post-transcriptional gene silencing and hence it is more appropriate or convenient to silence the pre-core gene of HBV or its equivalent in another pathogen. However, the general techniques can be used to up-regulate expression such as by increasing gene copy numbers or antagonizing inhibitors of gene expression.

The terms “nucleic acids”, “nucleotide” and “polynucleotide” include RNA, cDNA, genomic DNA, synthetic forms and mixed polymers, both sense and antisense strands, and may be chemically or biochemically, modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

Antisense polynucleotide sequences, for example, are useful in silencing transcripts of the pre-core protein/HBeAg-encoding pre-core gene. Expression of such an antisense construct within a cell interferes with pre-core protein/HBeAg gene transcription and/or translation. Furthermore, co-suppression and mechanisms to induce RNAi such as using short interfering RNA (siRNA) or ONA-derived RNAi (ddRNAi) may also be employed. Hence, antisense or sense molecules may be directly administered. In this latter embodiment, the antisense or sense molecules may also be formulated in a composition and then administered by any number of means to target cells or administered via an expression construct.

A variation on antisense and sense molecules involves the use of morpholinos, which are oligonucleotides composed of morpholine nucleotide derivatives and phosphorodiamidate linkages (for example, Summerton and Weller, Antisense and Nucleic Acid Drug Development. 7: 187-195, 1997). Such compounds are injected into embryos and the effect of interference with mRNA is observed.

In one embodiment, the present invention employs compounds such as oligonucleotides and similar species for use in modulating the function or effect of nucleic acid molecules such as those encoding a pre-core protein/HBeAg, i.e. the oligonucleotides induce pre-transcriptional or post-transcriptional gene silencing. This is accomplished by providing oligonucleotides which specifically hybridize to, or have complementing with a nucleic acid molecule encoding the pre-core protein/HBeAg. The oligonucleotides may be provided directly to a cell or generated within the cell. As used herein, the terms “target nucleic acid” and “nucleic acid molecule encoding a pre-core protein/HBeAg gene transcript” have been used for convenience to encompass DNA encoding the pre-core protein/HBeAg, RNA (including pre-mRNA and mRNA or portions thereof) transcribed from such DNA, and also cDNA derived from such RNA. The hybridization of a compound of the subject invention with its target nucleic acid is generally referred to as “antisense” or may be part of a complex with dicer such as a RISC.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense or RNAi compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e. under physiological conditions in the case of in vivo assays or therapeutic treatment, and under conditions in which assays are performed in the case of in vitro assays.

“Complementary” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

In the context of the subject invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compounds of this invention, the present invention comprehends other families of compounds as well, including but not limited to oligonucleotide analogs and mimetics such as those herein described.

The open reading frame (ORF) or “coding region” which is known in the art to refer to the region between the translation initiation codon and the translation termination codon, is a region which may be effectively targeted. Within the context of the present invention, one region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may, therefore, fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Specific examples of preferred antisense or RNAi compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

The antisense or RNAi oligonucleotides may be administered by any convenient means including by inhalation, local or systemic means.

In an alternative embodiment, genetic constructs including DNA vaccines are used to generate antisense or ddRNAi molecules in vivo.

Following identification of an agent which interacts with pre-core protein/HBeAg or modulates a TLR or TLR pathway, it may be manufactured and/or used in a preparation, i.e., in the manufacture or formulation or a composition such as a medicament, pharmaceutical composition or drug. These may be administered to individuals in a method of treatment or prophylaxis of infection. Alternatively, they may be incorporated into a patch or slow release capsule or implant.

Thus, the present invention extends, therefore, to a pharmaceutical composition, medicament, drug or other composition including a patch or slow release formulation comprising a modulator of pre-core protein/HBeAg or TLR activity or gene expression or the activity or gene expression of a component of the TLR signaling pathway.

Another aspect of the present invention contemplates a method comprising administration of such a composition to a subject such as for treatment or prophylaxis of an infection or other disease condition. Furthermore, the present invention contemplates a method of making a pharmaceutical composition comprising admixing a compound of the instant invention with a pharmaceutically acceptable excipient, vehicle or carrier, and optionally other ingredients. Where multiple compositions are provided, then such compositions may be given simultaneously or sequentially. Sequential administration includes administration within nanoseconds, seconds, minutes, hours or days. Preferably, sequential administration is within seconds or minutes.

Multi-part including two-art pharmaceutical compositions or packs are also contemplated comprising multiple components such as those which interact with pre-core protein/HBeAg activity or levels and which modulates a TLR such as TLR-2 or TLR-4 together. Further anti-pathogen agents may also be included such as nucleoside analogs. Such multi-part pharmaceutical compositions or packs may maintain different agents or groups of agents separately. These are either dispensed separately or admixed prior to being dispensed.

Accordingly, another aspect of the present invention contemplates a method for the treatment or prophylaxis of an infection or other disease condition in a subject, said method comprising administering to said subject an effective amount of a compound as described herein or a composition comprising same.

Preferably, the subject is a mammal such as a human or an animal model system such as a mouse, rat, rabbit, guinea pig, hamster, zebrafish or amphibian or avian species such as a duck.

This method also includes providing a wild-type or mutant target gene function to a cell. This is particularly useful when generating an animal model. Alternatively, it may be part of a gene therapy approach. A target gene or a part of the gene may be introduced into the cell in a vector such that the gene remains extrachromosomal. In such a situation, the gene will be expressed by the cell from the extrachromosomal location. If a gene portion is introduced and expressed in a cell carrying a mutant target allele, the gene portion should encode a part of the target protein. Vectors for introduction of genes both for recombination and for extrachromosomal maintenance are known in the art and any suitable vector may be used. Methods for introducing DNA into cells such as electroporation calcium phosphate co-precipitation and viral transduction are known in the art. This aspect of the present invention extends to constructs which encode ddRNAi.

Gene transfer systems known in the art may be useful in the practice of genetic manipulation. These include viral and non-viral transfer methods. A number of viruses have been used as gene transfer vectors or as the basis for preparing gene transfer vectors, including papovaviruses (e.g. SV40, Madzak et al., J. Gen. Virol. 73: 1533-1536, 1992), adenovirus (Berkner, Curr. Top. Microbiol. Immunol. 158: 39-66, 1992; Berkner et al., BioTechniques 6; 616-629, 1988; Gorziglia and Kapikian, J. Virol. 66: 4407-4412, 1992; Quantin et al., Proc. Natl. Acad. Sci. USA 89: 2581-2584, 1992; Rosenfeld et al., Cell 68: 143-155, 1992; Wilkinson et al., Nucleic Acids Res. 20: 2233-2239, 1992; Stratford-Perricaudet et al., Hum. Gene Ther. 1: 241-256, 1990; Schneider et al., Nature Genetics 18: 180-183, 1998), vaccinia virus (Moss, Curr. Top. Microbiol. Immunol. 158: 25-38, 1992; Moss, Proc. Natl. Acad. Sci. USA 93: 11341-11348, 1996), adeno-associated virus (Muzyczka, Curr. Top. Microbiol. Immunol. 158: 97-129, 1992; Ohi et al., Gene 89: 279-282, 1990; Russell and Hirata, Nature Genetics 18: 323-328, 1998), herpesviruses including HSV and EBV (Margolskee, Curr. Top., Microbiol. Immunol. 158: 67-95, 1992; Johnson et al., J. Virol. 66: 2952-2965, 1992; Fink et al., Hum. Gene Ther. 3: 11-19, 1992; Breakefield and Geller, Mol. Neurobiol. 1: 339-371, 1987; Freese et al., Biochem. Pharmacol. 40: 2189-2199, 1990; Fink et al., Ann. Rev. Neurosci. 19: 265-287, 1996), lentiviruses (Naldini et al., Science 272: 263-267, 1996), Sindbis and Semliki Forest virus (Berglund et al., Biotechnology 11: 916-920, 1993) and retroviruses of avian (Bandyopadhyay and Temin, Mol. Cell. Biol. 4: 749-754, 1984; Petropoulos et al., J. Viol. 66: 3391-3397, 1992], murine [Miller, Curr. Top. Microbiol. Immunol. 158: 1-24, 1992; Miller et al., Mol. Cell. Biol. 5: 431-437, 1985; Sorge et al., Mol. Cell. Biol. 4: 1730-1737, 1984; and Baltimore, J. Virol. 54: 401-407, 1985; Miller et al., J. Virol. 62: 4337-4345, 1988] and human [Shimada et al., J. Clin. Invest. 88: 1043-1047, 1991; Helseth et al., J. Virol. 64: 2416-2420, 1990; Page et al., J. Virol. 64: 5270-5276, 1990; Buchschacher and Panganiban, J. Virol. 66: 2731-2739, 1982] origin.

Non-viral gene transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated gene transfer can be combined with direct in vivo gene transfer using liposome delivery, allowing one to direct the viral vectors to particular cells. Alternatively, the retroviral vector producer cell line can be injected into particular tissue. Injection of producer cells would then provide a continuous source of vector particles.

In an approach which combines biological and physical gene transfer methods, plasmid DNA of any size is combined with a polylysine-conjugated antibody specific to the adenovirus hexon protein and the resulting complex is bound to an adenovirus vector. The trimolecular complex is then used to infect cells. The adenovirus vector permits efficient binding, internalization and degradation of the endosome before the coupled DNA is damaged. For other techniques for the delivery of adenovirus based vectors, see U.S. Pat. No. 5,691,198.

Liposome/DNA complexes have been shown to be capable of mediating direct in vivo gene transfer. While in standard liposome preparations the gene transfer process is non-specific, localized in vivo uptake and expression have been reported in tumor deposits, for example, following direct in situ administration.

If the polynucleotide encodes a sense or antisense polynucleotide or a ribozyme or DNAzyme, expression will produce the sense or antisense polynucleotide or ribozyme or DNAzyme. Thus, in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

Cells which carry mutant target genes (e.g. pre-core protein/HBeAg or TLR-2 or TLR-4) can be used as model systems to study the effects of infection or other disease condition.

The compounds, agents, medicaments, nucleic acid molecules and other target antagonists or agonists of the present invention can be formulated in pharmaceutical compositions which are prepared according to conventional pharmaceutical compounding techniques. See, for example, Remington's Pharmaceutical Sciences, 18^{th} Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. topical, intravenous, oral, intrathecal, epineural or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, International Patent Publication No. WO 96/11698.

For parenteral administration, the compound may dissolved in a pharmaceutical carrier and administered as either a solution of a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered and the rate and time-course of administration will depend on the nature and severity of the condition being treated. Prescription of treatment, e.g. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, supra.

Alternatively, targeting therapies may be used to deliver the active agent more specifically to certain types of cell, by the use of targeting systems such as antibodies or cell specific ligands or specific nucleic acid molecules. Targeting may be desirable for a variety of reasons, e.g. if the agent is unacceptably toxic or if it would otherwise require too high a dosage or if it would not otherwise be able to enter the target cells.

Instead of administering these agents directly, they could be produced in the target cell, e.g. in a viral vector such as described above or in a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target cells. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application No. 0 425 731A and International Patent Publication No. WO 90/07936.

The present invention further contemplates diagnostic protocols such as to determine the presence or absence of infection or other disease condition, whether an infection has become chronic, the susceptibility of a subject to infection and/or the efficacy of a therapeutic protocol.

Immunological based pre-core protein/HBeAg or TLR detection protocols may take a variety of forms. For example, a plurality of antibodies may be immobilized in an array each with different specificities to particular pre-core protein/HBeAg or TLRs or monocytes or hepatocytes comprising pre-core protein/HBeAg or TLRs. Cells from a biopsy are then brought into contact with the antibody array and a diagnosis may be made as to the level and type of pre-core protein/HBeAg or TLRs elevated or down-regulated on or in the cell.

Other more conventional assays may also be conducted such as by ELISA, Western blot analysis, immunoprecipitation analysis, immunofluorescence analysis, immunochemistry analysis or FACS analysis.

The present invention provides, therefore, a method of detecting in a pre-core protein/HBeAg or a TLR or cell comprising same or fragment, variant or derivative thereof comprising contacting the sample with an antibody or fragment or derivative thereof and detecting the level of a complex comprising said antibody and the HBeAg or TLR or fragment, variant or derivative thereof compared to normal controls wherein altered levels of the pre-core protein/HBeAg or TLR is indicative of the presence or absence of infection or other disease condition.

Preferably, the TLR is TLR-2 and/or TLR-4.

As discussed above, any suitable technique for determining formation of the complex may be used. For example, an antibody according to the invention, having a reporter molecule associated therewith, may be utilized in immunoassays. Such immunoassays include but are not limited to radioimmunoassays (RIAs), enzyme-linked immunosorbent assays (ELISAs) and immunochromatographic techniques (ICTs), Western blotting which are well known to those of skill in the art. For example, reference may be made to Coligan et al., 1991-1997, supra which discloses a variety of immunoassays which may be used in accordance with the present invention. Immunoassays may include competitive assays. It will be understood that the present invention encompasses qualitative and quantitative immunoassays.

Suitable immunoassay techniques are described, for example, in U.S. Pat. Nos. 4,016,043, 4,424,279 and 4,018,653. These include both single-site and two-site assays of the non-competitive types, as well as the traditional competitive binding assays. These assays also include direct binding of a labeled antigen-binding molecule to a target antigen. The antigen in this case is the TLR or a fragment thereof.

Two-site assays are particularly favoured for use in the present invention. A number of variations of these assays exist, all of which are intended to be encompassed by the present invention. Briefly, in a typical forward assay, an unlabeled antigen-binding molecule such as an unlabeled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labeled with a reporter molecule capable of producing a detectable signal is then added and incubated, allowing time sufficient for the formation of another complex of antibody-antigen-labeled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labeled antibody are added simultaneously to the bound antibody. These techniques are well known to those skilled in the art, including minor variations as will be readily apparent.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labeled antibody that binds, as determined by the associated reporter molecule, is proportional to the amount of antigen bound to the immobilized first antibody.

An alternative method involves immobilizing the antigen in the biological sample and then exposing the immobilized antigen to specific antibody that may or may not be labeled with a reporter molecule. Depending on the amount of target and the strength of the reporter molecule signal, a bound antigen may be detectable by direct labelling with the antibody. Alternatively, a second labeled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:—
(a) direct attachment of the reporter molecule to the antibody;
(b) indirect attachment of the reporter molecule to the antibody; i.e., attachment of the reporter molecule to another assay reagent which subsequently binds to the antibody; and
(c) attachment to a subsequent reaction product of the antibody.

The reporter molecule may be selected from a group including a chromogen, a catalyst, an enzyme, a fluorochrome, a chemiluminescent molecule, a paramagnetic ion, a lanthanide ion such as Europium (Eu³⁴), a radioisotope including other nuclear tags and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in U.S. Pat. Nos. 4,366,241, 4,843,000, and 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by International Patent Publication No. WO 93/06121. Reference also may be made to the fluorochromes described in U.S. Pat. Nos. 5,573,909 and 5,326,692. Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodate. As will be readily recognized, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labeled antibody is added to the first antibody-antigen complex, allowed to bind, and then the excess reagent washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic colour visually detectable with a light microscope. The fluorescent-labeled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art and are particularly useful for the present method. However, other reporter molecules, such as radioisotope, chemiluminescent or bioluminescent molecules may also be employed.

Monoclonal antibodies to a pre-core protein/HBeAg or TLR may also be used in ELISA-mediated detection of the TLR. This may be undertaken in any number of ways such as immobilizing anti-pre-core protein/HBeAg or anti-TLR antibodies to a solid support and contacting these with liver cells. Labeled anti-pre-core protein/HBeAg or anti-TLR antibodies are then used to detect immobilized pre-core protein/HBeAg or TLR. Alternatively, antibodies to other liver cell surface markers are used. This assay may be varied in any number of ways and all variations are encompassed by the present invention. This approach enables rapid detection and quantitation of pre-core protein/HBeAg or TLR levels.

The subject antibodies are also useful in in situ hybridization analysis such as of biopsy material. Such analysis enables the rapid diagnosis of levels of pre-core protein/HBeAg or TLRs such as TLR-2 and TLR-4.

Preferably, the diagnostic assay is based on FACS or a Western blot procedure.

In another embodiment, the method for detection comprises detecting the level of expression in a cell of a polynucleotide encoding a pre-core protein/HBeAg or a TLR. Expression of such a polynucleotide may be determined using any suitable technique. For example, a labeled polynucleotide encoding a pre-core protein/HBeAg or TLR may be utilized as a probe in a Northern blot of an RNA extract obtained from the cell. Preferably, a nucleic acid extract from the animal is utilized in concert with oligonucleotide primers corresponding to sense and antisense sequences of a polynucleotide encoding the kinase, or flanking sequences thereof, in a nucleic acid amplification reaction such as RT PCR. A variety of automated solid-phase detection techniques are also appropriate. For example, a very large scale immobilized primer arrays (VLSIPS®) are used for the detection of nucleic acids as, for example, described by Fodor et al. (Science. 251: 767-777, 1991) and Kazal et al. (Nature Medicine. 2: 753-759, 1996). The above genetic techniques are well known to persons skilled in the art.

For example, for a pre-core protein/HBeAg or TLR encoding RNA transcript, RNA is isolated from a cellular sample suspected of containing pre-core protein/HBeAg or TLR RNA. RNA can be isolated by methods known in the art, e.g. using TRIZOL® reagent (GIBCO-BRL/Life Technologies, Gaithersburg, Md.). Oligo-dT, or random-sequence oligonucleotides, as well as sequence-specific oligonucleotides can be employed as a primer in a reverse transcriptase reaction to prepare first-strand cDNAs from the isolated RNA. Resultant first-strand cDNAs are then amplified with sequence-specific oligonucleotides in PCR reactions to yield an amplified product.

“Polymerase chain reaction” or “PCR” refers to a procedure or technique in which amounts of a preselected fragment of nucleic acid, RNA and/or DNA, are amplified as described in U.S. Pat. No. 4,683,195. Reference to “PCR” includes multiplexing PCR. Generally, sequence information from the ends of the region of interest or beyond is employed to design oligonucleotide primers. These primers will be identical or similar in sequence to opposite strands of the template to be amplified. PCR can be used to amplify specific RNA sequences and cDNA transcribed from total cellular RNA. See generally Mullis et al. (Quant. Biol. 51: 263, 1987; Erlich, eds., PCR Technology, Stockton Press, NY, 1989). Thus, amplification of specific nucleic acid sequences by PCR relies upon oligonucleotides or “primers” having conserved nucleotide sequences wherein the conserved sequences are deduced from alignments of related gene or protein sequences, e.g. a sequence comparison of mammalian TLR genes. For example, one primer is prepared which is predicted to anneal to the antisense strand and another primer prepared which is predicted to anneal to the sense strand of a cDNA molecule which encodes a pre-core protein/HBeAg or TLR.

To detect the amplified product, the reaction mixture is typically subjected to agarose gel electrophoresis or other convenient separation technique and the relative presence of the pre-core protein/HBeAg or TLR specific amplified DNA detected. For example, pre-core protein/HBeAg or TLR amplified DNA may be detected using Southern hybridization with a specific oligonucleotide probe or comparing is electrophoretic mobility with DNA standards of known molecular weight. Isolation, purification and characterization of the amplified pre-core protein/HBeAg or TLR DNA may be accomplished by excising or eluting the fragment from the gel (for example, see references Lawn et al., Nucleic Acids Res. 2: 6103, 1981; Goeddel et al., Nucleic Acids Res. 8: 4057-1980), cloning the amplified product into a cloning site of a suitable vector, such as the pCRII vector (Invitrogen), sequencing the cloned insert and comparing the DNA sequence to the known sequence of pre-core protein/HBeAg or TLR. The relative amounts of pre-core protein/HBeAg or TLR mRNA and cDNA can then be determined.

Real-time PCR is particularly useful in determining transcriptional levels of PCR genes. Determination of transcriptional activity also includes a measure of potential translational activity based on available mRNA transcripts. Real-time PCR as well as other PCR procedures use a number of chemistries for detection of PCR product including the binding of DNA binding fluorophores, the 5′ endonuclease, adjacent liner and hairpin oligoprobes and the self-fluorescing amplicons. These chemistries and real-time PCR in general are discussed, for example, in Mackay et al., Nucleic Acids Res. 30(6): 1292-1305, 2002; Walker, J. Biochem. Mol. Toxicology. 15(3): 121-127, 2001; Lewis et al., J. Pathol. 195: 66-71, 2001.

The present invention further provides gene arrays and/or gene chips and/or RNAse protection to screen for the up- or down-regulation of mRNA transcripts. This aspect of the present invention is particularly useful in identifying conditions which result in the down-of HBeAg transcripts or regulation of TLR gene transcripts.

In an additional method, extracellular cytokines produced or blocked as a direct or indirect result of TLR signalling may be screened. Examples of suitable cytokines includes TNF-α, IFN-α, IFN-β and IFN-γ. Conveniently, three cytokines are screened in serum, whole blood, urine or other body fluid. Even levels of extracellular or intracellular HBeAg may be measured.

The present invention is further described by the following non-limiting Examples.

### EXAMPLE 1

### Measurement of TLR2 and TLR4 Levels

Methods

HBV Baculovirus Infected HepG2

HepG2 cells were infected with HBV 1:3 wildtype, HBV 1:3 Precore mutant or mock baculovirus infected and grown for 7 days prior to harvesting and staining for flow cytometry. Some cells were reserved for total RNA extraction using the RNeasy mini kit (Qiagen) following the manufacturers specifications.

Flow Cytometry

Cell surface staining was performed on HepG2 cells using TLR2-FITC (TL2.1; eBioscience) and TLR4-PE (HTA125; eBioscience) antibodies. Appropriate isotype controls were used. Dead cells were gated out based on their scatter profile. Experiments were carried out on a FACSCalibur flow cytometer (BD). A total of 10000 cells were acquired for each sample. Data was analysed using FlowJo software (Tree Star Inc.). Relative fluorescence intensity was determined by subtracting the geometric mean fluorescence intensity of the mock infected cells from the wildtype or precore mutant infected cells.

QPCR

Total RNA was reversed transcribed using random hexamers prior to real time PCR analysis of the cDNA. PCR was performed in triplicate using TaqMan Universal PCR Master Mix and Assays-On-Demand Gene Expression Assay probes and primers (Applied Biosystems) in a final 10 μl volume. Signal detection was via ABI Prism 7700 sequence detection system programmed to 50° C., 2 min; 95° C. 10 min; 40 cycles of 95° C., 15 sec; 60° C., 1 min. The threshold cycle (C_{T}), values of each gene were compared with the C_{T} value of 18S (ΔC_{T}) and relative expression units (REU) calculated for each sample. Hence, REU=2´C_{T}(gene of interest)−C_{T}(18S)=2´ΔC_{T}

Results

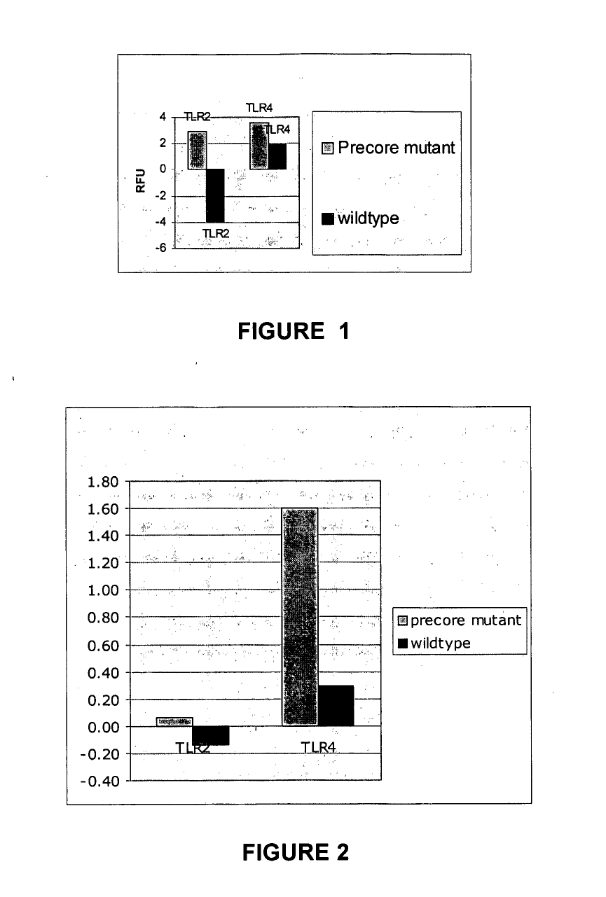

The results are shown FIGS. 1 and 2 and in Table 3. Levels of TLR2 and TLR4 are compared in HBV wild type versus pre-core mutant HBV infected cells.

TABLE 3

| | 30/04/2004 | | | | | |
|---|---|---|---|---|---|---|
| | TLR2 | | | TLR4 | | |
| Sample | Geomean Fluoresence | | | Geomean Fluorescence | | |
| uninfect | 8.83 | −1.08 | | 9.79 | 1.84 | |
| uninfectic | 9.91 | | | 7.95 | | |
| mockMOI10 | 8.64 | −0.12 | 0 | 9.49 | 2.25 | 0 |
| mockMOI10ic | 8.76 | | | 7.24 | | |
| mockMOI100 | 8.69 | 0.14 | 0 | 8.71 | 1.4 | 0 |
| mockMOI100ic | 8.55 | | | 7.31 | | |
| wtMOI10 | 9.85 | −1.15 | −1.03 | 10.4 | 3.46 | 1.21 |
| wtMOI10ic | 11 | | | 6.94 | | |
| wtMOI100 | 10.3 | 0 | −0.14 | 10.5 | 1.69 | 0.29 |
| wtMOI100ic | 10.3 | | | 8.81 | | |
| pcMOI10 | 8.81 | −0.69 | −0.57 | 8.23 | −0.03 | −2.28 |
| pcMOI10ic | 9.5 | | | 8.26 | | |
| pcMOI100 | 10.3 | 0.2 | 0.06 | 11.5 | 3 | 1.6 |
| pcMOI100ic | 10.1 | | | 8.5 | | |

### EXAMPLE 2

### Impaired TLR Expression in Chronic HBV Infection

Patients

Liver Biopsy

Single pass liver biopsies were performed on 5 patients with CHB. These were clinically stable patients attending a specialist liver outpatient clinic of a university teaching hospital. They had normal or mildly elevated transaminases (average ALT 87.8 U/L (N<45); average AST 32.2 U/L (N<45). Ishak modified histological activity index scores varied between 1/18-7/18, with disease stages between 1/6-3/6. Four of the five patients were HBeAg positive and had ongoing viral replication (HBV DNA 200—1.1×10⁸ copies/ml median 1500 copies/ml). Biopsies were placed in RPMI-1640 (Gibco-BRL) for transport to the laboratory where single cell suspensions were performed. Half of the biopsy (1.5×8 mm) was subjected to either a wire mesh or glass homogensizer with a loose pistol in order to separate about 6×10⁴ hepatocytes mixed with other cells. No collagenase or DNAse was used in this process in order to prevent receptor damage. This single cell suspension was then stained with appropriate antibodies and analysed by flow cytometry (see below).

Hepatitis B Virus Reagents and In Vitro Model Cell Culture Systems

Cell Cultures

The human hepatoblastoma cell line HepG2 was maintained in Minimum Essential Medium, (MEM; Invitrogen/Gibco) supplemented with penicillin and streptomycin, L-Glutamine and 10% v/v heat inactivated foetal calf serum (FCS) (Invitrogen). All cell lines were passaged weekly, and maintained at 37° C. in 5% v/v CO₂, with media changed every three days.

Human hepatoma (Huh-7) cells were maintained in Dulbecco's modified Eagle medium (Gibco-BRL, Grand Island, N.Y.) supplemented with 10% heat-inactivated FCS, 100 U/ml penicillin G, and 100 U/ml streptomycin (Gibco-BRL).

The human hepatoma cell line PLC/PRF/5 release HBsAg in the form of 22 nm particles predominantly with occasional filaments into the cell culture supernatant (Alexander et al, S. Afr Med J 54(23):973-974, 1978). These cells are grown and maintained in MEM with 10% FCS and do not produce HBV. The cell line was obtained (Mycoplasma free) from the American Type Culture Collection (ATCC: CTL −8024). Cell culture supernatant from PLC/PRF/5 cells was collected 5 days after seeding and contained high levels of HBsAg (>1 ug/ml) as demonstrated by enzyme immunoassay (IMX: Abbott Laboratories, North Chicago, Ill.).

Hepatitis B Virus Production by Transient Transfection

Recombinant HBV was generated by transfecting HepG2 cells with an infectious cDNA clone of HBV using Fugene 6 reagent (Roche, Ind.) (Chen et al, Hepatology 27(1):27-35, 2003) and after five days the cell culture supernatant was harvested. The supernatant containing secreted virus was collected, pooled and centrifuged to pellet cell debris. HBV was then concentrated by ultracentrifugation of the supernatant using the SW28 rotor (Beckman) through a 20% sucrose cushion (20% w/v sucrose, 1 mM EDTA, 30 mM Tris pH7.4, 150 mM NaCl) for 16 hours at 25,000 rpm, at 12° C.

To quantitate the virus in the pelleted material, a 20 μl aliquot of concentrated HBV was extracted for DNA using the MagNA Pure (Trade Mark) extraction system according to the manufacturer's instructions. HBV DNA was then quantitated in the Light-Cycler (Roche) using the ARTUS real time PCR kit with the titre expressed in viral genome copies/ml according to manufacturer's specifications. The sample was also tested for HBsAg and HBeAg by standard enzyme immunoassays (IMX: Abbott Laboratories, North Chicago, Ill.).

HBV Precore and Core Protein Producing Cell Lines

Huh-7 cells with tight inducible expression of the HBV precore and core protein were produced by cloning the HBV core or precore genes from genotype D cDNA (Chen 2003 Supra) into the tetracycline responsive expression system (pTRE-2; Clontech, Palo Alto, Calif.). The establishment and characterisation of these three cell lines PC47 (Precore producing), C4B (Core producing) and Parent (control cell line) has been published in detail (Locarnini et al., J Clin Virol. 32:113-21, 2005). Cell culture supernatant was collected after day 10 of culture in the presence (repressed protein expression) or absence (induced protein expression) of tetracycline.

Recombinant HBV Baculovirus Transduced Cells

Recombinant HBV baculovirus constructs were generated by site-directed mutagenesis and co-transfection, using a 1.3 genome length wildtype (WT) HBV template (genotype D, subtype ayw) (Invitrogen, Stratagene, Calif.), as previously described (Chen 2003 Supra). HepG2 cells were then transduced in parallel with control, WT, and precore mutant recombinant HBV baculovirus at a multiplicity of infection (MOI) of 50 plaque forming units (PFU) per cell (22, 23). Cell culture media was changed on days 1, 3, and 5 post-transduction and cells harvested on day 7.

In Vitro HBV Stimulation of Whole Blood Cultures

Five hundred microlitres of whole lithium-heparin blood was diluted with 500 μl RPMI-1640 supplemented with antibiotics and 5% v/v heat-inactivated fetal bovine serum and incubated at 37° C. with gentle rotation in tightly capped 5 ml polystyrene tubes (Becton Dickinson, San Jose, Calif.). Cells were stimulated with HBV wildtype 1.5 (Genotype A) at concentrations of 1, 10 and 50×10⁶ viral genome copies per ml, 1 μg/ml lamivudine, cell supernatant from PLC/PRF/5 cells (HBsAg) or supernatant from HBV precore (pC47: HBeAg-positive) or core protein (C4B) producing cell lines as well as appropriate control cell lines. After 20 hours, culture supernatants were collected and stored at −20° C. until cytokine analysis. The remaining cells were stained for flow cytometry.

TNF-α ELISAs

TNF-α was measured by capture ELISA using OptEIA set (Becton Dickinson, San Jose, Calif.) according to the manufacturer's specifications. Sensitivity of the ELISA was 8 pg/ml.

Flow Cytometry

Liver Cell Suspension

Cell surface staining was performed on liver cell suspensions derived from the patients's liver biopsy using CD14-PerCP (MφP9; Becton Dickinson, San Jose, Calif.), TLR2-FITC (TL2.1; eBioscience, San Diego, Calif.) and TLR4-PE (HTA125; eBioscience, San Diego, Calif.). Appropriate isotype controls were used. Cells were gated according to the amount of CD14 surface expression; CD14 high or CD14 low. This correlated with their scatter profile. CD14 high cells (hepatocytes) were much larger and more granular than CD14 low cells (Kupffer cells).

Patient Blood

Cell surface staining was performed on fresh lithium-heparin blood using CD14-PerCP (MφP9; Becton Dickinson, San Jose, Calif.), TLR2-FITC (TL2.1; eBioscience, San Diego, Calif.) and TLR4-PE (HTA125; eBioscience, San Diego, Calif.) as described previously (Riordan et al., Hepatology 37:1154-64, 2003) Appropriate isotype controls were used. Based on their scatter profile, monocytes were gated picking up the lymphocyte tail on a FACSCalibur flow cytometer (Becton Dickinson, San Jose, Calif.). A total of 8000 CD14+ monocytes were acquired for each sample. Data was analysed using FlowJo software (Tree Star Inc., Ashland, Oreg.). Relative fluorescence intensity was determined as a ratio of the geometric mean fluorescence intensity of the sample over its isotype matched control. Results expressed as % change of TLR.

Whole Blood Cultures

Cell surface staining was performed on whole blood cultures using CD14-PerCP (MφP9; Becton Dickinson, San Jose, Calif.), TLR2-FITC (TL2.1; eBioscience, San Diego, Calif.) and TLR4-PE (HTA125; eBioscience, San Diego, Calif.). Appropriate isotype controls were used. Based on their scatter profile, monocytes were gated picking up the lymphocyte tail on a FACSCalibur flow cytometer (Becton Dickinson, San Jose, Calif.). A total of 8000 CD14+ monocytes were acquired for each sample. Data were analysed using FlowJo software (Tree Star Inc., Ashland, Oreg.). Relative fluorescence intensity was determined as a ratio of the geometric mean fluorescence intensity of the sample over its isotype matched control, relative to the unstimulated or Parent stimulated control. Results expressed in % change in TLRs.

Human Hepatoma Cell Lines

To optimize cell condition and receptor integrity for subsequent flow cytometry, all the human liver cell line monolayers were washed in Hank's balanced salt solution (calcium, magnesium-free) 5 times, before incubating in Versene solution (0.02% w/v EDTA.4Na mixed 1:1 with Hanks balanced salt solution) to detach the cell monolayer. Serum-free MEM was then used to make a single cell suspension, before staining for flow cytometry. Cells were then stained with TLR2-FITC (TL2.1; eBioscience, San Diego, Calif.) and TLR4-APC (HTA125; eBioscience, San Diego, Calif.) immediately after cell harvest at room temperature. Cells were fixed with PBS plus 1% v/v formalin and run immediately after staining.

Quantitative PCR

RNA was isolated from cell lines using the RNeasy MiniKit (Qiagen) according to the manufacturers specifications. RNA was eluted from the column into 50 ul of nuclease free water and stored at −70° C. The concentration of RNA was estimated spectrophotometrically at OD 260 nm.

cDNA was synthesized from lug of RNA using random hexamers. The cDNA samples were stored at −70° C.

TaqMan Real time PCR (QPCR) was performed in 384-well plates using the Assays-On-Demand Gene Expression Products (Applied Biosystems) and an ABI Prism 7900HT Sequence Detection System (Applied Biosystems). The relative amounts of PCR product were determined using the comparative Ct method, where the amount of target DNA was normalised to 18s ribosomal RNA and relative to the mock cDNA (2^{-deltadeltaCT}).

Results

Peripheral Blood and Hepatic Cells Show Similar Downregulation of TLR2

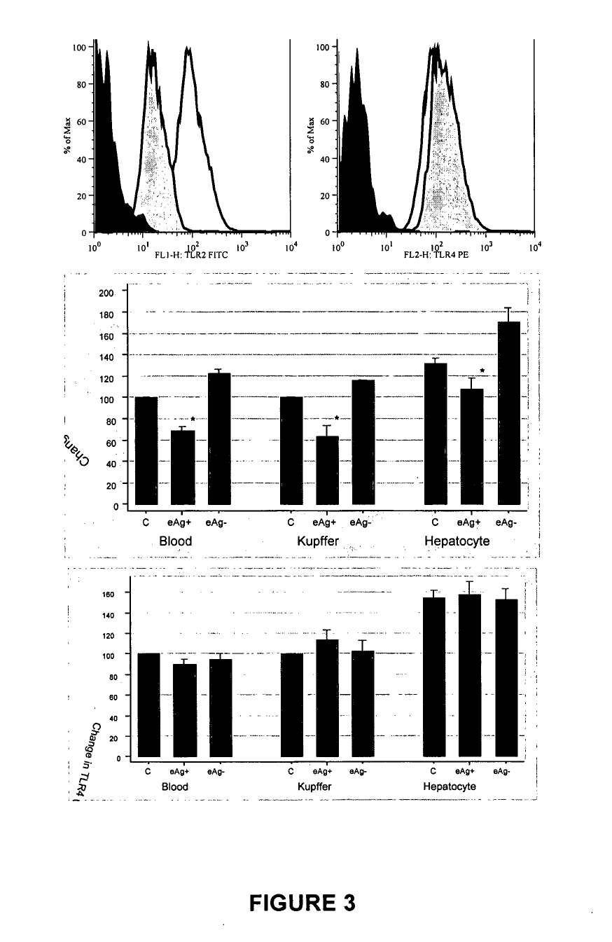

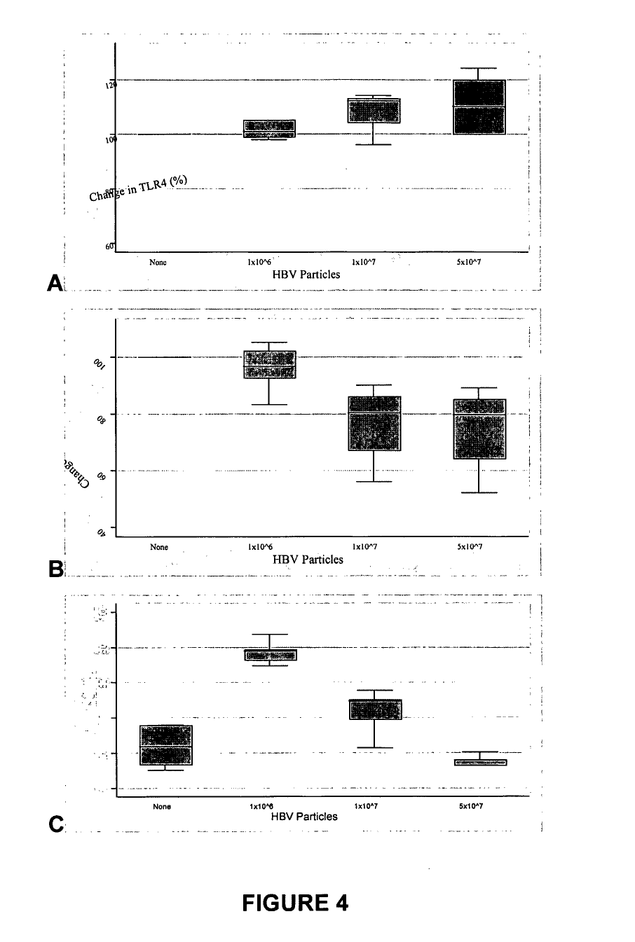

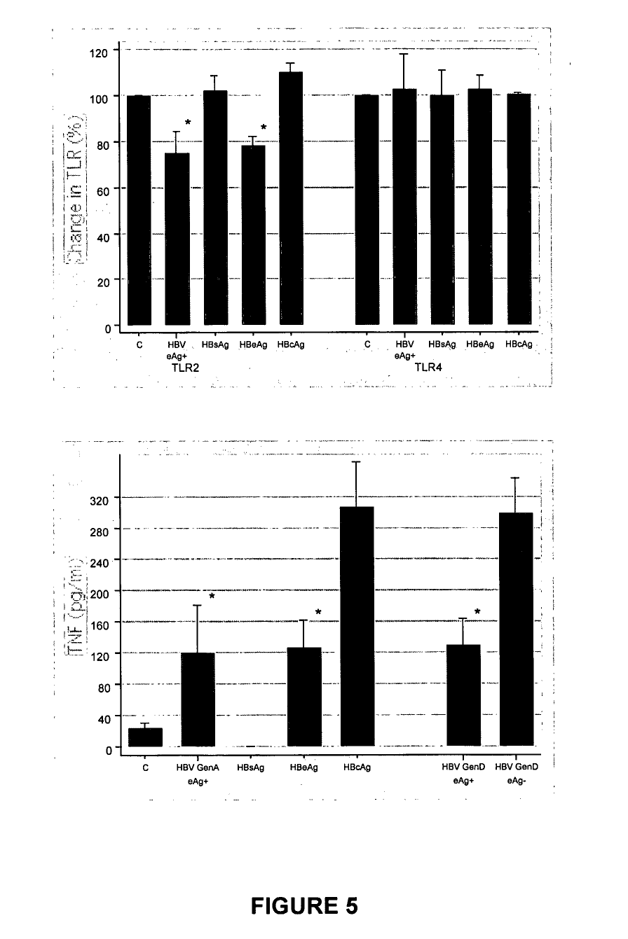

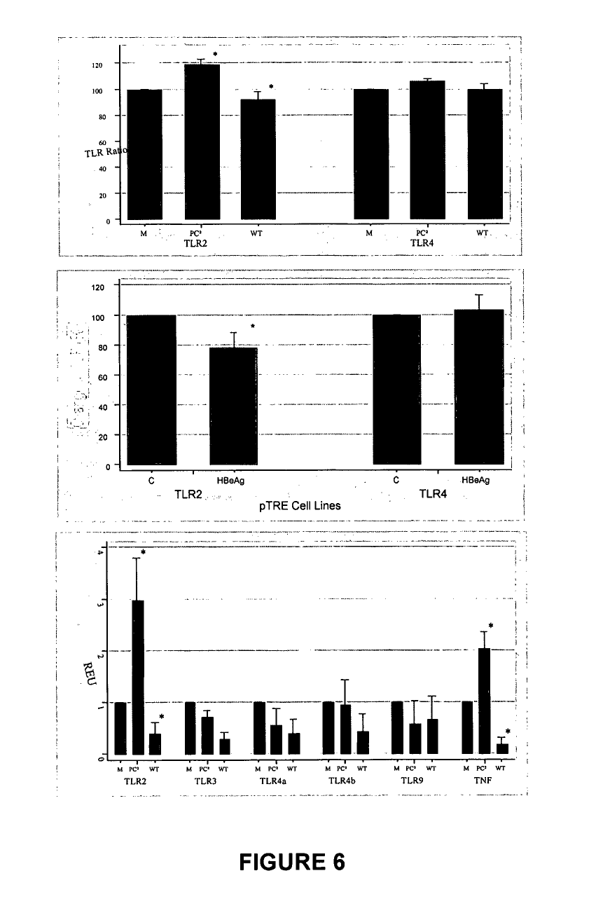

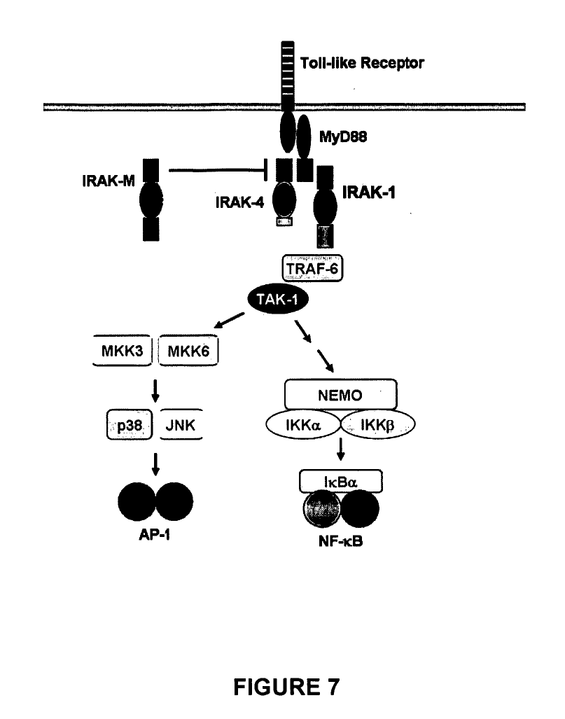

Peripheral blood and hepatocytes were examined from liver biopsies from four patients with HBeAg-positive CH-B, three patients with HBeAg-negative CH-B and five steatosis controls. The hepatocytes from these liver biopsies were separated into single cell suspensions as described in the Methods section and two separate CD14+ve populations of cells were gated by flow cytometry as seen in FIG. 3. In these two populations, (one which represented hepatocytes and one which represented Kupffer cells), TLR2 and TLR4 were measured (FIGS. 4 and 5). TLR2 was also detected on hepatocytes and moreover, its level of expression was downregulated on hepatocytes from HBeAg positive CHB as compared to HBeAg negative CHB and steatosis patients (FIG. 5). This downregulation of TLR2 confirms the previous demonstration in peripheral blood was also apparent on both hepatocytes and Kupffer cells of HBV infected patients (FIG. 5) (Visvanathan et al, GUT 52:130, 2003). The level of TLR4 expression did not differ significantly between the three groups.

Baculovirus Constructs in HepG2 cells

In order to investigate further the interaction of the precore protein and the TLR pathway observed clinically we used the recombinant HBV baculovirus transduction system of HepG2 cells was used in vitro. HepG2 cells were transduced in parallel with control, wild type or precore mutant recombinant HBV baculovirus and after 7 days were processed and stained for expression of TLR2 and TLR4 by flow cytometry as outlined previously (FIG. 4).

The transduced cells were also processed for RNA extraction in order to quantitated, by real time PCR the level of mRNA of TLR2, TLR3, TLR4 (two transcript variants) TLR9 and TNF-α (FIG. 6). The geometric mean fluorescence of TLR2 and TLR4 was expressed as a ratio to that of the isotype control antibody and the data presented as percent change in TLR.

The results of these flow cytometric experiments established that the precore mutant virus (HBeAg-negative) transduced HepG2 cells had significantly increased levels of TLR2 in comparison to wildtype virus (HBeAg-positive) transduced HepG2 cells. The level of TLR4 expression did not alter significantly between the three groups. These results were confirmed on repeated experimentation.

The relative amounts of PCR product for each transcript is shown in FIG. 6 (lower panel) and was determined using the comparative C_{T} method where the amount of target DNA was normalised to 18S rRNA and relative to the mock cDNA as outlined in the methods. This data represents the mean and standard error of three separate experiments. The striking feature is the upregulation of TLR2 mRNA with the precore mutant (HBeAg-negative) transduced cells compared to the profound downregulation of the transcript in the wild-type HBeAg-positive transduced cells, both with respect to the control cells. Recombinant HBV baculovirus transduced cells also resulted in a downregulation of TLR3, TLR4 and TLR9 irrespective of HBeAg status (FIG. 6). Most importantly the corresponding diminished production of TNF-α at both the message (FIG. 6) and protein levels is a functional correlate of the reduction in TLR2.

Stimulation of Whole Blood with HBV and Its Components.

We next examined the in vitro stimulation of whole blood with varying dilutions of HBV and its individual antigenic components. After 20 hours of incubation the blood was analysed for expression of TLR2 and TLR4 on CD14 positive monocytes by flow cytometry (FIG. 5). In addition the supernatants were harvested from these stimulations and TNF-α was measured by ELISA (FIG. 6). Exposure of CD14+ cells to HBV resulted in prominent suppression of TLR2 but not TLR4 further confirming the clinical data obtained in patients with chronic HBeAg-positive HBV infection. TNF-α suppression paralleled the decrement in TLR2 indicating that there was a functional correlation of the decreased TLR in these stimulation experiments. The precore protein stimulation also demonstrated a similar parallel suppression of TNF-α that has been shown with wild type virus indicating that this may be the factor that downregulates TLR2 in CHB.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Lien, E. & Golenbock, D. T. Nat Immunol 4, 1162-4, 2003
Pasare, C. & Medzhitov, R. Curr Opin Immunol 15, 677-82, 2003.
Leadbetter, E. A. et al. Nature 416, 603-7 (2002). Adams et al., Cancer Res. 53: 4026-4034, 1993
Akashi et al., J Immunol. 164: 3471-3475, 2000
Akira, S. J Biol Chem 278, 38105-8, 2003
Alexander et al, S. Afr Med J 54(23):973-974, 1978
Baltimore, J. Virol. 54: 401-407, 1985
Bandyopadhyay and Temin, Mol. Cell. Biol. 4: 749-754, 1984
Barton, G. M. & Medzhitov, R. Science 300, 1524-5, 2003
Beutler, B., et al., J Leukoc Biol 74, 479-85; 2003
Beckebaum et al, Immunology. 109; 487-495, 2003
Berglund et al., Biotechnology. 11: 916-920, 1993
Berkner et al., BioTechniques. 6; 616-629, 1988
Berkner, Curr. Top. Microbiol. Immunol. 158: 39-66, 1992
Bieback et al., J Virol. 76: 8729-8736, 2002
Boni et al, J Clin Invest. 102:968-75, 1998
Breakefield and Geller, Mol. Neurobiol. 1: 339-371, 1987
Buchschacher and Panganiban, J. Virol. 66: 2731-2739, 1982
Chen et al, Hepatology 27(1):27-35, 2003
Chothia et al., J. Mol. Biol. 196: 901, 1987
Chothia et al., J. Mol. Biol. 227: 799, 1992
Coligan et al., “Current Protocols in Immunology, John Wiley & Sons, Inc., 1991-1997
Cumber et al., J. Immunol. 149: 120-126, 1992
Davies and Riechmann, FEBS Lett. 339: 285-290, 1994
Erickson et al., Science. 249: 527-533, 1990
Ferrari et al, J Immunol. 145:3442-9, 1990
Fink et al., Ann. Rev. Neurosci. 19: 265-287, 1996
Fink et al., Hum. Gene Ther. 3: 11-19, 1992
Fodor et al., Science 251: 767-777, 1991
Freese et al., Biochem. Pharmacol. 40: 2189-2199, 1990
Gefter et al., Somatic Cell Genet. 3: 231-236, 1977
Genesca et al., Am J Gastroenterol. 94: 169-177, 1999
Glockshuber et al., Biochem. 29: 1363-1367, 1990
Goeddel et al., Nucleic Acids Res. 8: 4057-1980
Gorziglia and Kapikian, J. Virol. 66: 4407-4412, 1992
Gunther et al, Adv Virus Res. 52:25-137, 1999
Haeberle et al., J Infect Dis. 186: 1199-1206, 2002
Hamers-Casterman et al., Nature. 363: 446-448, 1993
Helseth et al., J. Virol. 64: 2416-2420, 1990
Hodgson, Bio/Technology. 9: 19-21, 1991
Hunt et al, Hepatology. 31(5):1037-44, 1994
Johnson et al., “Peptide Turn Mimetics” in Biotechnology and Pharmacy, Pezzuto et al., Eds., Chapman and Hall, New York, 1993
Johnson et al., J. Virol. 66: 2952-2965, 1992
Jones et al., Nature. 321: 522-525, 1986
Jung et al, Virology. 261:165-72, 1999
Kabat et al., “Sequences of Proteins of Immunological Interest”, U.S. Department of Health and Human Sciences, 1983
Kazal et al., Nature Medicine. 2: 753-759, 1996
Kennet et al., Monoclonal Antibodies and Hybridomas: A New Dimension in Biological Analyses, pp 376-384, Plenum Press, New York, 1980
Kohler and Milstein, Eur. J. Immunol. 6(7): 511-519, 1976
Köhler and Milstein, Nature. 256: 495-499, 1975
Kostelny et al., J. Immunol. 148: 1547-1553, 1992
Kozbor et al., Methods in Enzymology. 121: 140, 1986
Krebber et al., J. Immunol. Methods. 201(1): 35-55, 1997
Ku & Schutz, Proc. Natl. Acad. Sci. USA 92: 6552-6556, 1995
Kurt-Jones et al., Nat. Immunol. 1: 398-401, 2000
Lawn et al., Nucleic Acids Res. 2: 6103, 1981
Lewis et al., J. Pathol. 195: 66-71, 2001
Liu et al., Proc. Natl. Acad. Sci. USA 84: 3439-3443, 1987
Locarnini et al., J Clin Virol. 32:113-21, 2005
Lok et al, Proc Natl Acad Sci. USA. 91(9):4077-81, 1994
Mackay et al., Nucleic Acids Res. 30(6): 1292-1305, 2002
Madzak et al., J. Gen. Virol. 73: 1533-1536, 1992
Maini et al, J Exp Med. 191:1269-80, 2000
Margolskee, Curr. Top., Microbiol. Immunol. 158: 67-95, 1992;
Matsumoto et al., Biochem Biophys Res Commun. 293: 1364-1369, 2002
Medzhitov et al., N Engl J Med. 343: 338-344, 2000
Milich et al, J Immunol. 160:2013-21, 1998
Miller et al., J. Virol. 62: 4337-4345, 1988
Miller et al., Mol. Cell. Biol. 5: 431-437, 1985
Miller, Curr. Top. Microbiol. Immunol. 158: 1-24, 1992
Moss, Curr. Top. Microbiol. Immunol. 158: 25-38, 1992
Moss, Proc. Natl. Acad. Sci. USA 93: 11341-11348, 1996
Mullis et al., Quant. Biol. 51: 263, 1987
Muzyczka, Curr. Top. Microbiol. Immunol. 158: 97-129, 1992;
Naldini et al., Science. 272: 263-267, 1996
O'Reilly et al., Biotechniques. 25: 824-830, 1998
Ohi et al., Gene. 89: 279-282, 1990;
Page et al., J. Virol. 64: 5270-5276, 1990
Petropoulos et al., J. Viol. 66: 3391-3397, 1992
Plückthun et al., In Antibody engineering: A practical approach, 203-252, 1996
Plünckthun, Biochem. 31: 1579-1584, 1992
Quantin et al., Proc. Natl. Acad. Sci. USA 89: 2581-2584, 1992
Reiter et al., Biochem. 33: 5451-5459, 1994
Reiter et al., Cancer Res. 54: 2714-2718, 1994
Reiter et al., J. Biol. Chem. 269: 18327-18331, 1994
Remington's Pharmaceutical Sciences, 18^{th} Ed., 1990, Mack Publishing, Company, Easton, Pa., U.S.A.
Riordan et al., Hepatology 37: 1154-64, 2003
Richmann et al., Nature. 332: 323-327, 1988
Rosenfeld et al., Cell. 68: 143-155, 1992
Russell and Hirata, Nature Genetics. 18: 323-328, 1998
Schneider et al., Nature Genetics. 18: 180-183, 1998
Shimada et al., J. Clin. Invest. 88: 1043-1047, 1991
Shulman et al., Nature. 276: 269-270, 1978
Sorge et al., Mol. Cell. Biol. 4: 1730-1737, 1984
Stratford-Perricaudet et al., Hum. Gene Ther. 1: 241-256, 1990
Summerton and Weller, Antisense and Nucleic Acid Drug Development 7: 187-195, 1997
Takeuchi et al., Immunity. 11: 443-451, 1999
Tapping et al., J Immunol. 165: 5780-5787, 2000
Toyama et al., Monoclonal Antibody, Experiment Manual”, published by Kodansha Scientific, 1987
Trowbridge, J. Exp. Med. 148(1): 313-323, 1978
Verhoeyen et al., Science. 239: 1534-1536, 1988
Visvanathan et al, GUT 52:130, 2003
Volk et al., J. Virol. 42(1): 220-227, 1982
Walker, J. Biochem. Mol. Toxicology. 15(3): 121-127, 2001
Ward et al., Nature. 341: 544-546, 1989
Webber et al., Mol. Immunol. 32: 249-258, 1995
Wells, Methods Enzymol. 202: 2699-2705, 1991
Wilkinson et al., Nucleic Acids Res. 20: 2233-2239, 1992
Winter and Milstein, Nature. 349: 293, 1991

1. A method for detecting the presence of infection by Hepatitis B virus (HBV) or a disease condition or a predisposition thereto, said method comprising determining the presence or absence of an HBV-specified effector molecule which modulates the level of Toll-like receptor (TLR) signalling wherein the presence or absence of the effector molecule or an elevated or reduced level of the TLR or a component within the TLR signalling pathway is indicative of infection by HBV or the presence of an associated disease condition or predisposition thereto.

2. The method of claim 1 wherein pre-core expression product is an intracellular form.

3. The method of claim 1 wherein the pre-core expression product is an extracellular form.

4. The method of claim 1 wherein the TLR is TLR-2 or a homolog thereof.

5. The method of claim 1 wherein the level of TLR signalling is determined by the level of TLR or TLR-2 which in turn is determined by the amount of mRNA encoding same.

6. The method of claim 4 wherein the level of intracellular or extracellular pre-core expression product or TLR is determined at the protein level.

7. A method for monitoring a response to a therapeutic protocol directed against infection by HBV or development of a disease condition said method comprising determining the level or activity of a HBV-specified effector molecule which modulates TLR signalling wherein the presence or absence of the effector molecule or an elevated or reduced level of the TLR or a component within the TLR signalling pathway is indicative of infection by HBV or the presence of an associated disease condition or predisposition thereto.

8. The method of claim 7 wherein the HBV is a pre-core and/or basal core promoter (BCP) mutant.

9. The method of claim 8 wherein the HBV-specified effector molecule is an intracellular or extracellular pre-core expression product.

10. The method of claim 7 wherein the TLR is TLR-2 or a homolog thereof.

11. The method of claim 7 wherein the level of TLR signalling is determined by the level of TLR or TLR-2 which in turn is determined by the amount of mRNA encoding same.

12. The method of claim 10 wherein the level of intracellular or extracellular pre-core expression product or TLR or homolog thereof is determined at the protein level.

13. A method of treating a subject infected with HBV or having a disease condition or having a predisposition thereto, said method comprising administering to said subject an effective amount of an agent including a vaccine which down-regulates the level of intracellular or extracellular pre-core expression product or up- or down-regulates the level of a TLR.

14. The method of claim 13 wherein the HBV is a pre-core and/or BCP mutant.

15. The method of claim 13 wherein the HBV-specified effector molecule is an intracellular or extracellular pre-core expression product.

16. The method of claim 13 wherein the TLR is TLR-2 or a homolog thereof.

17. The method of claim 13 wherein the level of intracellular or extracellular pre-core expression product or TLR is determined by the amount of mRNA encoding same.

18. The method of claim 13 wherein the level of intracellular or extracellular pre-core expression product or TLR is determined at the protein level.

19. A pharmaceutical composition or vaccine for use in treating infection by HBV or a disease condition or a predisposition thereto, said composition comprising an modulator of intracellular or extracellular pre-core expression product and optionally a modulator of a TLR or a homolog thereof and one or more pharmaceutically acceptable carriers and/or diluents.
